SEQUENCE LISTING (31 sequences)

SEQ ID NO: 1 - DNA, 21 bp, Artificial Sequence
  Primer
aagtgggtgggagagttcgag

SEQ ID NO: 2 - DNA, 21 bp, Artificial Sequence
  Primer
aaggagccccttcgcctcgtg

SEQ ID NO: 3 - DNA, 21 bp, Artificial Sequence
  Primer
aacgatgccacctacggcaag

SEQ ID NO: 4 - DNA, 21 bp, Artificial Sequence
  Primer
aattctttggcctgaataatt

SEQ ID NO: 5 - DNA, 27 bp, Artificial Sequence
  Primer
agatacttcatcattcctcctcttttt

SEQ ID NO: 6 - DNA, 22 bp, Artificial Sequence
  Primer
ttgatatggcaattcctgcatt

SEQ ID NO: 7 - DNA, 18 bp, Artificial Sequence
  Primer
aggagaaatggtaggcaa

SEQ ID NO: 8 - DNA, 25 bp, Artificial Sequence
  Primer
ggtggtgcagatgaacttcagggtc

SEQ ID NO: 9 - DNA, 22 bp, Artificial Sequence
  Primer
ttgatatggcaattcctgcatt

SEQ ID NO: 10 - DNA, 22 bp, Artificial Sequence
  Primer
tgggatttccattgatgacaag

SEQ ID NO: 11 - DNA, 26 bp, Artificial Sequence
  Primer
ggatgcattgaggagaaatggtaggc

SEQ ID NO: 12 - DNA, 28 bp, Artificial Sequence
  Primer
ggcaggcctacaatacatactttattag

SEQ ID NO: 13 - DNA, 21 bp, Artificial Sequence
  Primer
aagtggggggaggggtcggca

SEQ ID NO: 14 - DNA, 23 bp, Artificial Sequence
  Primer
gcacttacctgtgttctggcggc

SEQ ID NO: 15 - DNA, 26 bp, Artificial Sequence
  Primer
gaaaaaaagaacgttcacggcgacta

SEQ ID NO: 16 - DNA, 22 bp, Artificial Sequence
  Primer
gcagtagtcgccgtgaacgttc

SEQ ID NO: 17 - DNA, 20 bp, Artificial Sequence
  Primer
aacgggtttgccgccagaac

SEQ ID NO: 18 - DNA, 29 bp, Artificial Sequence
  Primer
ggcgccgtccaggcacctcgattagttct

SEQ ID NO: 19 - DNA, 35 bp, Artificial Sequence
  Primer
aacttcagggtcagcttgccgtaggtggcatcgcc

SEQ ID NO: 20 - DNA, 29 bp, Artificial Sequence
  Primer
ggcgccgtccaggcacctcgattagttct

SEQ ID NO: 21 - DNA, 35 bp, Artificial Sequence; identical to SEQ ID NO: 19
  Primer

SEQ ID NO: 22 - DNA, 25 bp, Artificial Sequence
  Primer
ttcgcaacgggtttgccgccagaac

SEQ ID NO: 23 - DNA, 20 bp, Artificial Sequence
  Primer
agcaaagaccccaacgagaa

SEQ ID NO: 24 - DNA, 15 bp, Artificial Sequence
  Primer
ggcggcggtcacgaa

SEQ ID NO: 25 - DNA, 22 bp, Artificial Sequence
  Primer
cgcgatcacatggtcctgctgg

SEQ ID NO: 26 - DNA, 27 bp, Artificial Sequence
  Primer
agatacttcatcattcctcctcttttt

SEQ ID NO: 27 - DNA, 22 bp, Artificial Sequence
  Primer
ttgatatggcaattcctgcatt

SEQ ID NO: 28 - DNA, 18 bp, Artificial Sequence
  Primer
aggagaaatggtaggcaa

SEQ ID NO: 29 - DNA, 23 bp, Artificial Sequence
  Primer
tggcaccgtcaaggctgagaacg

SEQ ID NO: 30 - DNA, 22 bp, Artificial Sequence
  Primer
tgggatttccattgatgacaag

SEQ ID NO: 31 - DNA, 18 bp, Artificial Sequence
  Primer
ccacccatggcaaattcc

(19) United States
(12) Patent Application Publication
(10) Pub. No.: US 2009/0215860 A1
(43) Pub. Date: Aug. 27, 2009

(54) COMPOSITIONS AND METHODS FOR REGULATING GENE TRANSCRIPTION

(75) Inventors: Kevin V. Morris, San Diego, CA (US); David J. Looney, Encinitas, CA (US); Steven E. Jacobsen, Agoura Hills, CA (US)

Correspondence Address: MORRISON & FOERSTER LLP, 12531 HIGH BLUFF DRIVE, SUITE 100, SAN DIEGO, CA, 92130-2040, US

(73) Assignee: The Regents of the University of California

(21) Appl. No.: 11/570,822

(22) PCT Filed: Jun. 17, 2005
(86) PCT No.: PCT/US05/21577

§ 371 Date: Nov. 21, 2008

Related U.S. Application Data
(60) Provisional application No. 60/580,773, filed on Jun. 17, 2004.

(51) Int. Cl. A61K 31/7105; C12Q 1/68; C12Q 1/70; C12N 5/00

(52) U.S. Cl. 514/44 A; 435/6; 435/5; 435/375

(57) ABSTRACT

The invention is directed to compositions and methods for RNA-mediated gene regulation, e.g., transcription regulation, e.g., by transcriptional silencing of genes. In one aspect, the invention provides methods using siRNAs directed at a transcription regulator, e.g., a promoter or enhance sequence, of a gene target molecule. In one aspect, this results in in vivo DNA methylation and/or modification of associated chromatin (e.g., histone proteins) accompanied by and/or partial or complete transcription gene silencing in a cell, such as a mammalian cell, e.g., a human cell.

### FEDERAL FUNDING LEGEND

This invention was produced in part using funds from the Federal government under NIH Grant Nos. AI07384 and AI45992; and NIH AIDS training grant AI07384, NIH ROI AI45992, and UCSD CFAR AI36214. Accordingly, the Federal government has certain rights in this invention.

### TECHNICAL FIELD

This invention relates to molecular and cellular biology, biochemistry, molecular genetics, gene therapy, and drug design and discovery. In one aspect, the invention is directed to compositions comprising short interfering RNAs (siRNAs) that silence genes (e.g. animal genes) at the transcriptional level and methods of using them. In another aspect, the invention is directed compositions and methods for the methylation of target sequences or associated chromatin (histone proteins). In another aspect, the invention is directed to transcriptional gene silencing or inhibition of gene transcription. In another aspect, the invention is directed to compositions and methods for gene therapy and drug design or discovery. For example, siRNAs of the invention can be used to target and partially or completely silence specific gene target molecules as part of a gene therapy protocol, and compounds that interact with siRNAs or target molecules can be used to discover or design drugs.

### BACKGROUND

Small nucleotide RNAs are involved in eukaryotic biological processes, including, but not limited to, transposon silencing and antiviral defense by short interfering RNAs (siRNAs) and developmental gene regulation by micro RNAs (miRNAs) (see, e.g., Tijsteman (2002) Annu. Rev. Genet. 36:489-519; Grewal (2003) Science 301:798-802; Bartel (2004) Cell 116:281-297). Small RNA-mediated transcriptional gene silencing was first observed in plants by using inverted repeat transgenes or transgenic viruses to generate siRNAs homologous to a target promoter (see, e.g., Mette (2000) EMBO J 19:5194-5201; Jones (2001) Current Biology 11:747-757; Sijen (2001) Current Biology 11:436-440). Although gene silencing in plants has revolutionized the art of plant biology, comparable advancements have not been made in RNA-mediated transcriptional gene silencing in vertebrate animals such as mammals. Thus, there is a need for transcriptional gene silencing in such animals.

Double-stranded RNA (dsRNA) can induce sequence-specific posttranscriptional gene silencing in many organisms by a process known as RNA interference (RNAi). RNA interference (RNAi) is a phenomenon wherein double-stranded RNA, when present in a cell, inhibits expression of a gene that has an identical or nearly identical sequence. Inhibition is caused by degradation of the messenger RNA (mRNA) transcribed from the target gene. The double-stranded RNA responsible for inducing RNAi is termed interfering RNA. The mechanism and cellular machinery through which dsRNA mediates RNAi has been investigated using both genetic and biochemical approaches. Biochemical analyses suggest that dsRNA introduced into the cytoplasm of a cell is first processed into RNA fragments 21-25 nucleotides long. It has been shown in in vitro studies that these dsRNAs, termed small interfering RNAs (siRNA), are generated at least in part by the so-called “Dicer” RNase III-like enzyme. These siRNAs likely act as guides for mRNA cleavage, as the target mRNA is cleaved at a position in the center of the region covered by a particular siRNA.

Interference of gene expression by siRNAs is now recognized as a naturally occurring strategy for silencing genes in C. elegans, Drosophila, plants, and in mouse embryonic stem cells, oocytes and early embryos (see, e.g., Baulcombe, Plant Mol. Biol., 32, 79 (1996); Kennerdell and Carthew, Cell, 95, 1017 (1998); Svoboda et al., Development, 127, 4147 (2000); Timmons and Fire, Nature, 395, 854 (1998); Waterhouse et al., Proc. Natl. Acad. Sci. U.S.A., 95, 13959 (1998); Wianny (2002) Nat. Cell Biol. 2:70; Yang et al., Mol. Cell Biol., 21, 7807 (2001)). However, in mammalian cells, dsRNA that is 30 base pairs or longer can induce sequence-nonspecific responses that trigger a shut-down of protein synthesis. Additionally, studies in mammalian cells involve post-transcriptional gene silencing. To promote the development of drugs for mammalian targets, it would be advantageous to study gene functions in mammals that involve sequence-specific responses and transcriptional gene expression.

siRNAs and miRNAs are processed from double-stranded RNA precursors by the conserved RNAse III/RNA helicase Dicer (see, e.g., Tijsterman (2002) Annu. Rev. Genet. 36:489-519). Argonaute proteins can bind small RNAs, and are components of downstream effector complexes that down-regulate gene expression by a variety of mechanisms (M. A. Carmell, Z. Xuan, M. Q. Zhang, G. J. Hannon, Genes Dev 16, 2733-42 (2002)). Small RNAs with perfect homology to their target can serve as a specificity guide for mRNA cleavage (called RNA interference in animals), while those with mismatches to their target mediate translational inhibition (D. P. Bartel, Cell 116, 281-97 (2004)). siRNAs directed against promoter sequences also cause transcriptional gene silencing in the yeast S. pombe, and transcriptional silencing in Drosophila has been linked to an Argonaute protein (M. Pal-Bhadra, U. Bhadra, J. A. Birchler., Molecular Cell 9, 315-327 (2002); and V. Schramke, R. Allshire, Science 301, 1069-74 (2003)). DNA methylation in the plant Arabidopsis thaliana can be guided by siRNAs, and maintenance of S. pombe centromeric heterochromatin depends on siRNA-directed histone H3 lysine 9 methylation (M. F. Mette, W. Aufsatz, J. Van der Winden, A. J. M. Matzke, and M. A. Matzke., The EMBO Journal 19, 5194-5201 (2000); M. Wassenegger, M. W. Graham, M. D. Wang., Cell 76, 567-576 (1994); S. W. Chan et al., Science 303, 1336 (2004); D. Zilberman, X. Cao, S. E. Jacobsen, Science 299, 716-9 (2003); and T. A. Volpe, C. Kidner, I. M. Hall, G. Telig, S. I. S. Grewal, R. A. Martienssen., Science 297, 1833-1837 (2002)).

Both histone modification (Cheung (2005) Molecular Endocrinology 19, 563-73) and cytosine DNA methylation are major mediators of epigenetic modulation of gene expression in mammalian cells, and de novo DNA methylation in plants is guided by small RNAs (Chan (2004) Science 303, 1336; Bird (1999) Cell 99, 451-4; and Matzke (2001) Science 293, 1080-3).

### SUMMARY

The invention provides methods and compositions (e.g., kits) for regulating gene transcription, e.g., silencing or down-regulating genes, in a cell, such as an animal cell, e.g., human cells. In one aspect, the invention provides RNA-mediated methods of transcriptional regulation of gene expression, e.g., silencing or down-regulating genes. The methods and compositions (e.g., kits) can be used to target and screen genes in animals and to design or discover drugs that inhibit or promote said gene silencing. The methods and compositions (e.g., kits) can also be used to treat patients with diseases, infections and conditions, e.g., of genetic origin, or any disease, infection or condition whose treatment, amelioration or prevention would benefit by regulating gene transcription, e.g., silencing or down-regulating a gene of interest. The targeted gene can be an endogenous gene, e.g., a gene encoding a dominant negative mutant, or a heterologous gene, e.g., an integrated virus, such as a lentivirus (e.g., HIV) integrated into a host's genome. The methods and compositions (e.g., kits) of the invention can also be used to upregulate expression of a gene, e.g., by silencing or down-regulating transcription of a negative regulator of a nucleic acid or polypeptide whose expression is desired to be upregulated, or, alternatively, by interfering with endogenous RNA-mediated regulatory pathways restricting expression of the target gene. In this later aspect, the siRNA of the invention would target nucleic acid involved in endogenous RNA-mediated regulatory pathways.

The invention provides methods and compositions (e.g., kits) for silencing genes comprising identifying a target sequence in a genome of a cell, e.g., an animal cell, such as a human cell; obtaining a target sequence specific siRNA; transfecting the target specific siRNA into a nucleus of the cell; and monitoring gene suppression in the cell. Gene silencing in the methods and compositions (e.g., kits) include transcriptional gene silencing, which may be partial or complete silencing (i.e., down-regulating). In one aspect of the invention, the target sequence can be a promoter or an enhancer region including intronic region enhancers. In one aspect of the invention, the target sequence can comprise marker or reporter genes as a means of monitoring gene expression. In another aspect, gene suppression is reversible, or alternatively irreversible. In one aspect, gene suppression by a method of the invention is constitutive, or alternatively, inducible. In another aspect of the invention, target sequences are amplified.

In one aspect of the invention, the siRNA comprises dsRNA generated by duplex RNA transcription (transcribing each strand of RNA separately to make dsRNA), stem-loop siRNA generated by transcription as stem-loop constructs, and/or siRNA generated by transcription of sequences targeting a nucleic acid sequence, e.g., a genomic sequence or a viral sequence, e.g., targeting promoters and/or enhancers, where the sequences targeting the nucleic acid are embedded within longer RNA message designed to be retained in the nucleus (e.g., embedding the siRNA within a longer transcript). In one aspect, the invention comprises delivery of a single-stranded inhibitory RNA directed at promoters and/or enhancer elements.

The invention provides methods and compositions (e.g., kits) for methylating target sequences or associated chromatin (e.g., histone proteins) or a combination thereof, e.g., genomic gene sequences encoding nucleic acids or polypeptides whose expression is desired to be down-regulated (silenced). In one aspect, the methods and compositions (e.g., kits) for methylating target nucleic acid sequences or associated chromatin include identifying a target sequence in a genome of a cell, e.g., an animal cell, such as a human cell; obtaining a target sequence specific siRNA; transfecting the target specific siRNA into a nucleus of the cell, wherein the transfection results in the methylation of the target sequence or associated chromatin; and analyzing the methylated target sequences.

The invention provides methods and compositions (e.g., kits) for in vivo DNA methylation of a gene or associated chromatin (e.g., histone proteins) or a combination thereof, comprising: (a) providing a target sequence specific short interfering RNA (siRNA), wherein the target sequence is complementary to a gene sequence in a cell, e.g., an animal cell, such as a human cell, and the cell comprising genomic nucleic acid comprising the target sequence; and (b) importing the target specific siRNA into the nucleus of the cell, thereby effecting in vivo DNA methylation of the gene target sequence or associated chromatin, e.g., effecting in vivo DNA methylation of histone proteins associated with the target sequence.

The invention provides methods and compositions (e.g., kits) for inhibiting gene silencing by identifying a target sequence in a genome of a cell, e.g., an animal cell, such as a human cell; obtaining a target sequence specific siRNA; transfecting target specific siRNAs into a nucleus of the cell; treating the cell with gene silencing inhibitors; and monitoring inhibition of gene suppression in the cell. Silencing inhibitors used in the methods of the invention include, but are not limited to, trichostatin A and 5-azacytidine, or equivalent compounds.

In one aspect of the methods, importing the target specific siRNA into the nucleus comprises permeabilization of the nuclear envelope of a cell, e.g., an animal cell, such as a human cell. In one aspect, permeabilization of the nuclear envelope is effected by a lentiviral (e.g., a vector comprising an HIV genome) co-transduction. In one aspect, the transfecting of the target specific siRNA into a nucleus comprises use of a nuclear active transport mechanism or a nuclear-transport mediating composition, which can be a polypeptide or peptide, comprising an importin β (karyopherin β), an SV40 T antigen nuclear localization signal, a human LEDGF/p75 protein, the nuclear localizing peptide NLSV404 (see, e.g., Ritter (2003) J. Mol. Med. 81:708-717, Epub 2003 Oct. 22), a tetramer of NLSV404 (of the nuclear localization signal (NLS) of the SV40 large T-antigen), the amphipathic peptide of 27 residues called “MPG” (which contains a hydrophobic domain derived from the fusion sequence of HIV gp41 and a hydrophilic domain derived from the nuclear localization sequence of SV40 T-antigen, see, e.g., Morris Nucleic Acids (1997) Res. 25:2730-2736; Deshayes (2004) Biochim. Biophys. Acta. 1667:141-147), a nucleoporin protein or an active fragment thereof or a combination thereof.

In one aspect, the transfecting of the target specific siRNA into a nucleus comprises use of a nuclear localization sequence, e.g., the siRNA can comprise one or more nuclear localization sequence(s), many of which are known in the art (see, e.g., Bremner (2004) Bioconjug. Chem. 15:152-161). In one aspect, the nuclear active transport mechanism or nuclear-transport mediating composition, or nuclear localization sequence, is encoded by the same expression system modality that encodes and/or expresses the siRNA used to practice the invention in vivo, ex vivo or in vitro, e.g., a mammalian artificial chromosome (MAC), a human artificial chromosome, a yeast artificial chromosome, a bacterial artificial chromosome (BAC), a P1 artificial chromosome or P1-derived vector (PACs), a cosmid, a recombinant virus, a phage, a vector or a plasmid.

In one aspect, the target nucleic acid sequence comprises a transcriptional regulatory sequence and the siRNA comprises a sequence completely, or partially, complementary to the transcriptional regulatory sequence. In one aspect, the transcriptional regulatory sequence comprises a promoter sequence and the siRNA comprises a sequence complementary to the promoter sequence. In one aspect, the transcriptional regulatory sequence comprises an enhancer sequence and the siRNA comprises a sequence complementary to the enhancer sequence.

The methods or compositions can use constructs which, in alternative aspects, express short sense and antisense RNA strands separately, in tandem, embedded within a transcript designed to be retained in the nucleus, or in short hairpin RNA transcripts designed to saturate or overcome nuclear export, and thus remain in the nucleus.

In one aspect, the siRNA of the invention is associated with a nucleic acid sequence, e.g., an RNA, such as a naturally retained RNA, that is retained in the nucleus. In one aspect, these nucleic acid sequences retained in the nucleus (e.g., naturally retained RNAs) comprise stem-loop structures, or they can be single antisense sequences, or they can be nuclear RNAs or small nuclear RNA. The siRNA of the invention can be associated with the nucleic acid sequences retained in the nucleus (e.g., naturally retained RNAs) directly or indirectly. For example, the siRNA and nucleic acid sequence retained in the nucleus can be encoded the same expression construct, or on different expression constructs. In another aspect, they can be associated with a composition, e.g., a peptide or polypeptide, that binds to a nucleic acid sequences retained in the nucleus (e.g., naturally retained RNAs), e.g., nuclear RNAs or small nuclear RNA.

In one aspect, the siRNA modifies gene transcription of one or more target genes. In one aspect, the siRNA partially silences, or down-regulates, transcription of the target gene, thereby effecting at least partial silencing of the gene. In one aspect, the siRNA completely silences, or down-regulates, transcription of the target gene, thereby effecting complete silencing of the gene.

In one aspect, partial or complete transcriptional suppression occurs prior to transcription and generation of mRNA. In some aspects, the transcriptional down-regulation (e.g., “gene silencing”) effected by the methods of the invention can be either transcriptional gene silencing (TGS), post-transcriptional gene silencing (PTGS) or a combination thereof. In one aspect, the at least partial silencing or down-regulation (or complete or down-regulation) of the gene is reversible, or alternatively, irreversible.

In one aspect, a method of the invention can comprise, or further comprise, in vivo, ex vivo or in vitro methylation of a target gene sequence and/or associated chromatin (e.g., histone proteins), which can be an endogenous gene sequence or a exogenous gene sequence, e.g., as an integrated provirus, such as a lentivirus (e.g., an integrated HIV genome). In one aspect, the target sequence comprises a marker or reporter gene.

In one aspect, the methods and compositions of the invention can be used to regulate transcription or silence genes in a mammalian cell, such as a human cell, or a tissue, or to an individual, which can be a human. The methods and compositions of the invention can be used in a veterinary context, e.g., on animals, such as wild, domesticated, laboratory or farm animals.

In one aspect, the methods and compositions of the invention can further comprising amplification of the target sequence, e.g., where the amplification can comprise a polymerase chain reaction amplification (PCR).

In one aspect, the methods and compositions of the invention can be use siRNA of any length, e.g., exemplary siRNA can be 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or 10 or fewer nucleotides in length. For example, in alternative aspects, an siRNA used in the methods and compositions of the invention can be 19 to 25 nucleotides in length, or, 21 to 23 nucleotides in length. In alternative aspects, a siRNA used in the methods and compositions of the invention can have at least a portion, most substantially all, or all of the siRNA comprising a double-stranded RNA. In one aspect, the at least partially or completely double-stranded siRNA comprises two separate oligonucleotides. In one aspect, the at least partially double-stranded siRNA can comprise any secondary structure, e.g., a folded oligonucleotide, e.g., a hairpin secondary structure.

In one aspect, the at least partially or completely double-stranded siRNA comprises a single stranded nucleotide overhang, e.g., wherein the single stranded nucleotide overhang comprises a 3′ single stranded nucleotide overhang, or a 5′ single stranded nucleotide overhang, or both. In one aspect, the single stranded nucleotide overhang comprises a two or three nucleotide overhang, e.g., 3′ overhang, 5′ overhang or both ends overhanging. In one aspect, the siRNA comprises a triple helix-forming oligonucleotide that specifically binds to a double-stranded DNA sequence. In one aspect, the siRNA can comprise an active sequence embedded within a longer RNA designed to avoid export from the nucleus.

In one aspect, the siRNA comprises a synthetic, non-natural or modified RNA. In one aspect, the synthetic, non-natural or modified RNA comprises 2′-O-methyl-containing ribonucleotide, a phosphorothioate-containing ribonucleotide, 2′-O-(2-methoxyethyl)-modified oligonucleotide, or a combination thereof. In one aspect, the synthetic, non-natural or modified RNA comprises a phosphodiester, a phosphorothioate or a 2′-O-methyl phosphodiester oligonucleotide, or a combination thereof.

The invention provides methods and compositions for methylating a genomic target sequences or associated chromatin (histone proteins) in a cell, e.g., an animal cell, such as a human cell, comprising: (a) identifying a target sequence in a genome of the cell; (b) providing a target sequence specific siRNA; (c) transfecting the target specific siRNA into a nucleus of the cell, wherein the transfection results in methylation of the target sequence or associated chromatin (histone proteins); and (d) analyzing the methylated target sequences or histone proteins.

The invention provides methods and compositions for methylating target sequences or associated chromatin (histone proteins) comprising: (a) providing a target sequence specific siRNA, wherein the target sequence is complementary to a gene sequence in the animal cell; and (b) importing the target specific siRNA into a nucleus of the animal cell. In one aspect, the method further comprises amplification of the target sequence.

The invention provides methods and compositions for inhibiting gene silencing in a cell comprising: (a) identifying a target sequence in a genome of the cell; (b) providing a target sequence specific siRNA; (c) transfecting the target specific siRNA into the nucleus of animal cell; (d) treating the animal cell with gene silencing inhibitors; and (e) monitoring inhibition of gene suppression in the animal cell. In one aspect, the gene silencing inhibitors comprise trichostatin A and 5-azacytidine.

The invention provides methods and compositions for ameliorating or treating a disease or a condition in a subject by transcriptional silencing comprising: (a) administering to the subject a composition comprising a siRNA that targets a gene of interest, wherein transcriptional silencing of the gene of interest treats or ameliorates the disease or condition; and (b) importing the gene targeting siRNA into the nucleus of a cell of the subject, wherein the cell comprises the gene of interest, thereby silencing expression of the gene of interest and ameliorating the disease or condition. In one aspect, the method partially or completely silences transcription of the gene of interest. In one aspect, the method further comprises integrating the gene of interest into a subject genome.

The invention provides methods and compositions for drug discovery comprising: (a) identifying a target gene sequence in a genome of an animal cell; (b) providing a gene target sequence specific siRNA; (c) importing the gene target specific siRNA into the nucleus of the animal cell, wherein the siRNA suppresses target gene expression; and (d) interacting a test agent with the animal cell or siRNA to determine which test agent inhibits or promotes gene silencing.

The invention provides methods and compositions for transcriptionally silencing a latent provirus or retrovirus comprising (a) providing a target sequence-specific short interfering RNA (siRNA), wherein the target sequence is complementary to at least one gene of a latent provirus or retrovirus gene sequence integrated into the genome of an animal cell; and (b) importing the target specific siRNA into a nucleus of the animal cell. In one aspect, the integrated provirus comprises an integrated retrovirus or lentivirus, such as a human immunodeficiency virus, e.g., HIV, e.g., HIV-1 or HIV-2. In one aspect, the target sequence is complementary to an LTR of the latent provirus or retrovirus, wherein optionally the LTR comprises an HIV-1 LTR.

The invention provides kits for transcriptionally silencing a latent provirus or retrovirus, the kit comprising siRNA or nucleic acid encoding siRNA, and instructions comprising instructions for practicing any one of the methods of the invention. The invention provides kits for transcriptionally silencing a gene, the kit comprising siRNA or nucleic acid encoding siRNA, and instructions comprising practicing any one of the methods of the invention. The invention provides kits for the targeted methylation of a gene or associated chromatin (histone proteins), the kit comprising siRNA, nucleic acid encoding siRNA, and instructions comprising instructions for practicing any one of the methods of the invention.

The invention provides methods and compositions for treating patients and methods of drug discovery. The method of treating patients provided herein includes administering to a patient a composition comprising a siRNA (or, alternatively, comprising a nucleic acid encoding the siRNA), wherein the siRNA targets a gene of interest; and transfecting the gene targeting siRNA (or, alternatively, a nucleic acid encoding the siRNA) into a nucleus of the patient's cell, wherein expression from the targeted gene of interest is reduced. The transfecting the gene targeting siRNA also includes transfection or infection by a vector, e.g., a viral vector, e.g., an adenovirus vector comprising a nucleic acid encoding the siRNA. A gene of interest can include, but is not limited to, a gene associated with cancer, with a viral infection, e.g., a retroviral infection, e.g., HIV infection, with neurodegenerative disorders, and with genetic disorders.

For example, siRNA used to practice the invention can target DNA viruses such as Epstein-Barr virus (EBV), cytomegalovirus (CMV), Rhesus monkey rhadinovirus (RRV), Kaposi's sarcoma-associated helpesvirus (KSHV), parvovirus B19 (B 19), varicella-zoster virus (VZV), and/or any or the herpesvirus, e.g., human herpesvirus (HHV)-1, (HHV)-2, (HHV)-5, (HHV)-6, (HHV)-7, or human herpesvirus 8 (HHV-8). siRNA used to practice the invention also can target Sindbis-like virus (e.g., Togaviridae, Bromovirus, Cucumovirus, Tobamovirus, Ilarvirus, Tobravirus, and Potexvirus), Picornavirus-like viruses (e.g., Picornaviridae, Caliciviridae, Comovirus, Nepovirus, and Potyvirus), minus-stranded viruses (e.g., Paramyxoviridae, Rhabdoviridae, Orthomyxoviridae, Bunyaviridae, and Arenaviridae), double-stranded viruses (e.g., Reoviridae and Birnaviridae), Flavivirus-like viruses (e.g., Flaviviridae and Pestivirus), Retrovirus-like viruses (e.g., Retroviridae), Coronaviridae, Nodaviridae, Flaviviridae, Filovirus, a Marburg or Ebola virus, a virus of the genus Flavivirus, such as yellow fever virus, dengue virus, West Nile virus, St. Louis encephalitis virus, Japanese encephalitis virus, Murray Valley encephalitis virus, Rocio virus, tick-borne encephalitis virus, and the like, to name only a few possible targets for siRNA of the invention. Thus, the invention provides methods for treating, ameliorating, or preventing infection any infectious agent, including any of these exemplary, or other viruses.

The method of drug discovery includes identifying a target sequence in a genome of an animal cell; obtaining a target sequence specific siRNA; transfecting the target specific siRNA into a nucleus of the animal cell, wherein the siRNA suppresses gene expression; and interacting drugs with the animal cell or siRNA to determine which drugs inhibit or promote gene silencing.

Also provided herein are animal cells (e.g., human cells) whose genes have been transcriptionally silenced by siRNAs using the methods and compositions of the invention. Also provided herein are kits and libraries of compounds for practicing the gene silencing methods provided herein.

The details of one or more embodiments of the invention are set forth in the accompanying drawings and the description below. Other features, objects, and advantages of the invention will be apparent from the description and drawings, and from the claims.

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

All publications, patents, patent applications, GenBank sequences and ATCC deposits, cited herein are hereby expressly incorporated by reference for all purposes.

### DESCRIPTION OF DRAWINGS

FIG. 1A shows Lentiviral FIV vector pVE-GFPwP integrating an EF1A promoter-GFP transgene into human cells, as described in detail in Example 1, below. FIG. 1B shows promoter-targeted siRNA inhibits gene expression, as described in detail in Example 1, below.

FIG. 2 shows data demonstrating de novo DNA methylation guided by siRNA using exemplary methods of the invention. FIG. 2A shows HinP1I-based DNA methylation assay of the EF1A promoter, as described in detail in Example 1, below. FIG. 2B shows TSA and 5-azaC counteract transcriptional inhibition caused by DNA methylation, as described in detail in Example 1, below. FIG. 2C shows Bisulfite genomic sequencing of the EF1A promoter from an integrated transgene, including the EF52 siRNA target site, as described in detail in Example 1, below.

FIG. 3A shows that using an exemplary method of the invention promoter-targeted siRNA inhibits endogenous EF1A, as described in detail in Example 1, below. FIG. 3B shows EF52 siRNA causes de novo DNA methylation of the endogenous EF1A promoter, as described in detail in Example 1, below. FIG. 3C shows Nuclear-imported siRNAs inhibit an integrated EF1A promoter-GFP transgene long after lentiviral transduction, as described in detail in Example 1, below.

FIG. 4 shows GFP expression as determined by FACS 72 hrs post-transduction, as described in detail in Example 1, below.

FIG. 5 shows the result of GAPDH real-time kinetic RT PCR from three independent measurements. siRNA mediated inhibition is specific, as described in detail in Example 1, below.

FIG. 6 shows that treatment with siRNA does not alter vector integration, as described in detail in Example 1, below.

FIG. 7 shows that siRNA transfection does not alter total copies of vector in transduced cells, as described in detail in Example 1, below.

FIG. 8 shows that endogenous EF1a expression is inhibited by EF52 in transduced siRNA transfected 293FT cells, as described in detail in Example 1, below.

FIG. 9A shows primers used to determine EF1a transcriptional profile relative to various features of the integrated pVE-GFPwP vector and EF1a expressed GFP transgene, as described in detail in Example 1, below. FIGS. 9B, 9C and 9D show that integrated vector EF1a promoter expressed GFP transgene is spliced correctly, as described in detail in Example 1, below.

FIG. 10 is an illustration of the EF1a promoter and intron A targets, as described in detail in Example 2, below.

FIG. 11A illustrates data showing intron targeted siRNA suppression of GFP using this exemplary method of the invention, as described in detail in Example 2, below. FIG. 11B illustrates data where 293FT cells were transduced with FIV packaged pVEGFPwP and later transfected with siRNAs, as described in detail in Example 2, below. FIG. 11C illustrates data where human PBMCs were transduced with FIV packaged pVEGFPwP and 24 hrs later transfected with siRNAs, as described in detail in Example 2, below.

FIG. 12 illustrates the specificity of transcriptional inhibition produced by using intron-targeted siRNAs in exemplary methods of the invention, as described in detail in Example 2, below.

Like reference symbols in the various drawings indicate like elements.

### DETAILED DESCRIPTION

The invention is directed to compositions and methods for RNA-mediated gene regulation, e.g., transcription regulation, e.g., by transcriptional silencing of genes. In one aspect, the invention provides methods using siRNAs directed at a transcription regulator, e.g., a promoter or enhancer sequence, of a gene target molecule. In one aspect, the results in methylation of DNA sequences and/or associated histones and/or partial or complete transcriptional gene silencing in a cell, such as a mammalian cell, e.g., a human cell.

The nucleic acids used in the methods of the invention can be synthetic, non-natural or modified RNA, e.g., nucleic acid comprising DNA, 2′-O-methyl-containing ribonucleotide, a phosphorothioate-containing ribonucleotide, 2′-O-(2-methoxyethyl)-modified oligonucleotide, or a combination thereof.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters. The term “gene” includes a nucleic acid sequence comprising a segment of DNA involved in producing a transcription product (e.g., a message), which in turn is translated to produce a polypeptide chain, or regulates gene transcription, reproduction or stability. Genes can include regions preceding and following the coding region, such as leader and trailer, promoters and enhancers, as well as, where applicable, intervening sequences (introns) between individual coding segments (exons).

The term “naturally occurring” is used to describe an object that can be found in nature as distinct from being artificially produced. For example, a protein or nucleotide sequence present in an organism (including a virus), which can be isolated from a source in nature and which has not been intentionally modified by a person in the laboratory, is naturally occurring.

The term “genome” refers to the complete genetic material of an organism.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells, and organisms comprising transgenic cells are referred to as “transgenic organisms”.

The terms “transformed”, “transduced”, “transgenic”, and “recombinant” refer to a host cell or organism into which a heterologous nucleic acid molecule has been introduced. The nucleic acid molecule can be stably integrated into the genome generally known in the art and are disclosed in Sambrook and Russell, infra. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

The terms “gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference. In some embodiments, gene silencing may be allele-specific. “Allele-specific” gene silencing refers to the specific silencing of one allele of a gene.

The term “RNA interference (RNAi)” refers to the process of sequence-specific, transcriptional gene silencing (e.g., posttranscriptional gene silencing) mediated or initiated by siRNA (or, “RNAi”). While the invention is not limited by any particular mechanism of action, during RNAi, in practicing the methods of the invention, siRNA can induce degradation of target mRNA with consequent sequence-specific inhibition of gene expression. In practicing the methods of the invention, siRNA also can induce in vivo genomic DNA methylation and/or methylation of associated proteins, e.g., histone proteins.

The phrases “small interfering” or “short interfering RNA” or siRNA also can refer to an RNA duplex of nucleotides, or, in some alternative aspects, a single molecule of RNA (which can, in some embodiments, have secondary structure, such as loops) that is targeted to a nucleic acid, e.g., a gene, of interest. A “RNA duplex” refers to the structure formed by the complementary pairing between at least two regions of a RNA molecule. Thus, the “RNA duplex” can comprise one, two, three or more RNA molecules. siRNA is “targeted” to a gene in that the nucleotide sequence of the duplex portion of the siRNA is complementary to a nucleotide sequence of the targeted gene. Thus, by using the sequence of a target gene, any siRNA can be routinely designed and made. In some embodiments, the length of the duplex of siRNAs is less than 30 nucleotides. In some embodiments, the length of the duplex of siRNAs is more than 30 nucleotides. In some embodiments, the duplex can be 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or 10 or less nucleotides in length. In some embodiments, the length of the duplex is 19-25 nucleotides in length. The RNA duplex portion of the siRNA can be part of a hairpin structure. In one aspect, there is no hairpin structure in an siRNA of the invention. In addition to the duplex portion, the hairpin structure may contain a loop portion positioned between the two sequences that form the duplex. The loop can vary in length. In some embodiments the loop is 2, 3, 4, 5, 6, 7, 8, 9, 10, 11, 12 or 13 or more nucleotides in length. The hairpin structure can also contain 3′ or 5′ overhang portions. In some embodiments, the overhang is a 3′ or a 5′ overhang 0, 1, 2, 3, 4 or 5 nucleotides in length. The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal. The siRNA can be entirely, or in part, comprised of synthetic nucleotides, natural bases or modified bases.

The term “treatment” as used herein refers to ameliorating at least one symptom of, curing and/or preventing the development of a disease or a condition.

The terms “transfection of cells” refer to the acquisition by a cell of new nucleic acid material by incorporation of added DNA. Thus, transfection refers to the insertion of nucleic acid into a cell using physical or chemical methods. Several transfection techniques are known to those of ordinary skill in the art including: calcium phosphate DNA co-precipitation; DEAE-dextran; electroporation; cationic liposome-mediated transfection (supra); and tungsten particle-facilitated microparticle bombardment (Johnston (1990)). Strontium phosphate DNA co-precipitation is also a transfection method.

The terms “transduction of cells” refer to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

As used herein, the terms “computer,” “computer program” and “processor” are used in their broadest general contexts and incorporate all such devices.

“Operably linked” as used herein refers to a functional relationship between two or more nucleic acid (e.g., DNA) segments. Typically, it refers to the functional relationship of transcriptional regulatory sequence to a transcribed sequence. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

As used herein, the term “promoter” includes all sequences capable of driving transcription of a coding sequence in a cell, e.g., a plant cell or animal cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

“Tissue-specific” promoters are transcriptional control elements that are only active in particular cells or tissues or organs, e.g., in plants or animals. Tissue-specific regulation may be achieved by certain intrinsic factors which ensure that genes encoding proteins specific to a given tissue are expressed. Such factors are known to exist in mammals and plants so as to allow for specific tissues to develop.

The term “overexpression” refers to the level of expression in transgenic cells or organisms that exceeds levels of expression in normal or untransformed cells or organisms.

The term “plant” includes whole plants, plant parts (e.g., leaves, stems, flowers, roots, etc.), plant protoplasts, seeds and plant cells and progeny of same. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous states. As used herein, the term “transgenic plant” includes plants or plant cells into which a heterologous nucleic acid sequence has been inserted, e.g., the nucleic acids and various recombinant constructs (e.g., expression cassettes) of the invention.

“Plasmids” can be commercially available, publicly available on an unrestricted basis, or can be constructed from available plasmids in accord with published procedures. Equivalent plasmids to those described herein are known in the art and will be apparent to the ordinarily skilled artisan.

The phrases “nucleic acid” or “nucleic acid sequence” includes oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoprotein (e.g., iRNPs). The term encompasses nucleic acids, i.e., oligonucleotides, containing known analogues of natural nucleotides, naturally occurring nucleic acids, synthetic nucleic acids, and recombinant nucleic acids. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antiseise Nucleic Acid Drug Dev 6:153-156.

“Amino acid” or “amino acid sequence” include an oligopeptide, peptide, polypeptide, or protein sequence, or to a fragment, portion, or subunit of any of these, and to naturally occurring or synthetic molecules. The terms “polypeptide” and “protein” include amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

The term “isolated” includes a material removed from its original environment, e.g., the natural environment if it is naturally occurring. For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

As used herein, the term “recombinant” can include nucleic acids adjacent to a “backbone” nucleic acid to which it is not adjacent in its natural environment. In one aspect, nucleic acids represent 5% or more of the number of nucleic acid inserts in a population of nucleic acid “backbone molecules.” “Backbone molecules” according to the invention include nucleic acids such as expression vectors, self-replicating nucleic acids, viruses, integrating nucleic acids, and other vectors or nucleic acids used to maintain or manipulate a nucleic acid insert of interest. In one aspect, the enriched nucleic acids represent 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more of the number of nucleic acid inserts in the population of recombinant backbone molecules. “Recombinant” polypeptides or proteins refer to polypeptides or proteins produced by recombinant DNA techniques; e.g., produced from cells transformed by an exogenous DNA construct encoding the desired polypeptide or protein. “Synthetic” polypeptides or protein are those prepared by chemical synthesis, as described in farther detail, below.

A promoter sequence can be “operably linked to” a coding sequence when RNA polymerase which initiates transcription at the promoter will transcribe the coding sequence into mRNA, as discussed farther, below.

“Oligonucleotide” includes either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide can ligate to a fragment that has not been dephosphorylated.

The phrase “substantially identical” in the context of two nucleic acids or polypeptides, can refer to two or more sequences that have, e.g., at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more nucleotide or amino acid residue (sequence) identity, when compared and aligned for maximum correspondence, as measured using one any known sequence comparison algorithm, as discussed in detail below, or by visual inspection.

A “substantially identical” amino acid sequence also can include a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an amylase, resulting in modification of the structure of the polypeptide, without significantly altering its biological activity. For example, amino- or carboxyl-terminal amino acids that are not required for amylase activity can be removed.

“Hybridization” includes the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

“Variant” includes polynucleotides or polypeptides of the invention modified at one or more base pairs, codons, introns, exons, or amino acid residues (respectively) yet still retain the biological activity of an amylase of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM and any combination thereof. Techniques for producing variant amylase having activity at a pH or temperature, for example, that is different from a wild-type amylase, are included herein.

### Gene Silencing

The invention provides a method of silencing or down-regulating genes in a cell, e.g., an animal cell, such as a human cell. In alternative aspects, the gene silencing is pre-transcriptional or post-transcriptional or combination thereof. In another aspect of the invention, the animals are mammals, such as humans.

Target Sequences

In one aspect of the method or composition of the invention for gene silencing or down-regulating, target sequences are first identified. Target sequences are sequences that are targeted, recognized, and/or bound by a siRNA used to practice the invention. Target sequences include, but are not limited to, nucleic acids or derivatives, variants, or portions thereof, including protein-encoding nucleic acids. The target sequence can be genomic, e.g., endogenous, or exogenous, e.g., a genome of an infectious agent, such as a viral, proviral or bacterial genome, etc. In one aspect of the invention, target sequences include any transcriptional regulatory sequence, e.g., promoter or enhancer sequences, or intronic or exonic sequences, or sequences responsible for the stability of conformation of a gene or transcript.

Promoters that can be targeted by a method or composition of the invention include, but are not limited to, CMV immediate early, HSV thymidine kinase, early and late SV40, LTRs from retrovirus, and mouse metallothionein I, heat shock promoters, and LTRs from retroviruses. Other promoters that can be targeted by a method or composition of the invention and known to control expression of genes in prokaryotic or eukaryotic cells or their viruses may also be used. In one aspect of the invention, the promoter is elongation factor 1 alpha (EF1A) promoter.

In another aspect of the invention, the target sequences comprise a reporter or marker gene. The reporter or marker gene is used to monitor gene expression. In particular, the reporter or marker gene is used to monitor gene suppression or silencing. In one aspect of the invention, the reporter gene is green fluorescent protein. Any compound, label, or gene that has a reporting or marking function can be used in the methods provided herein.

In another aspect of the invention, target sequences are inserted into the genome of a host cell by, e.g., a vector or a virus. In one aspect, the target sequence itself of a vector or a virus. In one aspect, the siRNA used to practice the invention is expressed from a vector, e.g., an expression vector, an expression cassette, a phage, a plasmid, a virus, an artificial chromosome and the like. A nucleic acid sequence (e.g., a target sequence, or nucleic acid encoding siRNA) can be inserted into a vector, expression cassette, plasmid, phage, virus, artificial chromosome, and the like, by a variety of procedures. In general, the sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which can be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell. In one aspect of the invention, target sequences are integrated into genomes using a lentiviral feline immunodeficiency (FIV) vector for the transduction process.

Obtaining siRNA and Target Sequences

The nucleic acids of the invention, including siRNA and nucleic acids that encode them, can be made, isolated and/or manipulated by, e.g., cloning and expression of cDNA libraries, amplification of message or genomic DNA by PCR, and the like. In practicing the methods of the invention, homologous gene expression can be modified (e.g., down-regulated or silenced) by targeting a template nucleic acid with an siRNA, as described herein. Alternatively, heterologous gene expression (e.g., viral, bacterial) can be modified (e.g., down-regulated or silenced) by targeting a template nucleic acid with an siRNA. The invention can be practiced in conjunction with any method or protocol or device known in the art, which are well described in the scientific and patent literature.

The nucleic acids used to practice this invention, whether RNA, siRNA, iRNA, siRNA, antisense nucleic acid, cDNA, genomic DNA, vectors, viruses or hybrids thereof, may be isolated from a variety of sources, genetically engineered, amplified, and/or expressed/generated recombinantly. Recombinant siRNA generated from these nucleic acids can be individually isolated or cloned and tested for a desired activity. Any recombinant expression system can be used, including bacterial, mammalian, yeast, insect or plant cell-based expression systems.

Alternatively, these nucleic acids (e.g., the siRNA—which can broadly also include DNA, synthetic, non-natural or modified RNA, e.g., 2′-O-methyl-containing or a phosphorothioate-containing ribonucleotide, or a combination thereof) can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Alternatively, nucleic acids can be obtained from commercial sources.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Nucleic acid used in the methods of the invention, e.g., modalities or vehicles for expressing the siRNA for practicing the methods of the invention in vivo, ex vivo or in vitro can comprise mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages, vectors or plasmids. The methods or compositions of the invention can use any of these expression systems, which can comprise nucleic acid that can express, inducibly or constitutively, siRNA useful for practicing the invention in vivo, ex vivo or in vitro.

In practicing the invention, nucleic acids of the invention or modified nucleic acids of the invention, can be reproduced by amplification. Amplification can also be used to clone or modify the nucleic acids of the invention. Thus, the invention provides amplification primer sequence pairs for amplifying nucleic acids of the invention. One of skill in the art can design amplification primer sequence pairs for any part of or the full length of these sequences.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; and Sooknanan (1995) Biotechnology 13:563-564.

### Cells

The methods and compositions of the invention can be practiced in vivo, ex vivo or in vitro. In one aspect, the invention provides cells whose gene expression has been down-regulated or silenced using the methods or compositions of the invention. In one aspect, the invention provides cells having an infectious or exogenous nucleic acid (e.g., a provirus) whose gene expression has been down-regulated or silenced using the methods or compositions of the invention. In one aspect, the invention provides cells comprising constructs that encode the siRNA of the invention, nucleic acid sequences retained in the nucleus (e.g., naturally retained RNAs), and e.g., stem-loop structures, single antisense sequences, nuclear RNAs or small nuclear RNA, or nucleic retention sequences, or sequences encoding nuclear retention peptides or polypeptides.

In one aspect of the invention, cells have gene expression that has been transcriptionally down-regulated or silenced. The cells whose genes have been transcriptionally down-regulated or silenced include bacterial, fungal, insect, plant and animal cells. Animal cells can include mammalian cells. Exemplary animal cells include CHO, COS or Bowes melanoma or any mouse or human cell line. The selection of an appropriate host is within the abilities of those skilled in the art.

Where appropriate, host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to interact with siRNAs. siRNAs siRNAs used in the methods and compositions of the invention can be obtained or made from a variety of sources, e.g., produced in vitro, ex vivo or in vivo, as described herein. In alternative embodiments, of the methods and compositions of the invention siRNAs can contain from about 1 to about 200 nucleotides, from about 5 to about 100 nucleotides, from about 10 to about 50 nucleotides, from about 15 to about 30 nucleotides, or from about 21 to about 25 nucleotides. siRNAs used in the methods and compositions of the invention can have perfect homology with target sequences to effect target specific responses. In another aspect of the invention, siRNAs used in the methods and compositions of the invention have about 99%, 98%, 97%, 96%, 95%, 94%, 92%, 91%, 90%, 88%, 86%, 84%, 82%, 80%, 78%, 76%, 74%, 72%, 70%, 65%, 60%, 55%, 50%, 45%, 40%, 35%, 30%, 25%, 20%, 15%, 10%, or 5%, homology with target sequences. In one aspect, siRNAs used in the methods and compositions of the invention can hybridize under physiologic conditions to a nucleic acid target sequence, e.g., they can specifically hybridize to a target sequence in a cell, e.g., in vivo. In another aspect of the invention, siRNAs target more than one target sequence, target marker or reporter gene.

The extent of sequence identity (homology) necessary for in vivo targeting of an siRNA to a target nucleic acid (e.g., specific binding of an siRNA to a target sequence in a cell under physiologic conditions) can be tested under routine screening conditions, e.g., in cell culture and the like.

The extent of sequence identity (homology) may be determined using any computer program and associated parameters, including those described herein, such as BLAST 2.2.2. or FASTA version 3.0t78, with the default parameters. Homology or sequence identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence, e.g., a sequence of the invention, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Methods of alignment of sequence for comparison are well known in the art. In alternative aspects, optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WD, or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Databases containing genomic information annotated with some functional information are maintained by different organization, and are accessible via the internet.

Entry of siRNAs into the Nucleus

In one aspect of the invention, target sequence specific siRNAs are designed to enter (pass through) nuclear membranes and thereby down-regulate or silence gene expression. In various aspects of the methods and compositions of the invention, entry of siRNA into the nucleus is designed to be effected by macromolecular transport processes across the nuclear envelope, vectors capable of transporting nucleic acids into a nucleus, e.g., a viral vector, such as a lentiviral vector, nuclear-transport mediating peptides, electroporation, lipid vesicles, MPG, or a combination thereof, All techniques known in the art can be used to practice the invention in vivo, ex vivo, or in vitro, e.g., see Morris, M. C., Vidal, P., Chaloin, L., Heitz, F. & Divita, G. A new peptide vector for efficient delivery of oligonucleotides into mammalian cells. Nucleic Acids Res 25, 2730-6 (1997)), including any transfecting agent known in the art, see, e.g., Davis, L., Dibner, M., Baftey, I., Basic Methods in Molecular Biology, (1986).

Macromolecular transport processes across the nuclear envelope are known in the art and a large number of soluble transport receptors mediating either nuclear import or nuclear export have been identified, and can be used to practice the invention. Most of these receptors belong to one large family of proteins, all of which share homology with the protein import receptor importin β (also named karyopherin β), which in one aspect is used to practice the invention. Members of this family have been classified as importins or exportins on the basis of the direction they carry their cargo. To date, the family includes 14 members in the yeast Saccharomyces cerevisiae and at least 22 members in humans, any of which can be used to practice the invention.

In addition to using importin β (karyopherin β) as macromolecular transport compositions, some aspects of the invention can comprise (use) SV40 T antigen nuclear localization signal, or, Human LEDGF/p75 protein, or, nucleoporins and transport factors. See, e.g., Yasuhara, Exp Cell Res. 2004 Jul. 1; 297(1):285-93; Maertens, J Biol Chem. 2004 May 25; Zolotukhin, J. Virol. 1999 January; 73(1):120-7.

### Gene Suppression

The invention provides siRNAs that can be used to silence or suppress gene expression. Provided herein are compositions and methods of gene suppression in which target sequence specific siRNAs interact with target sequences and suppress gene expression. In one aspect of the invention, gene suppression is transcriptional gene expression. In particular, siRNAs enter the nuclear membrane of host cells and specifically target a sequence of interest. Marker or reporter genes and compounds can be used to monitor gene expression. Other methods and assays known in the art, including but not limited to computer-based methods, can be used to monitor gene expression. In one aspect of the invention, siRNAs resulted in 93% reduction in transcription (see Example). However, any amount of reduction in transcription or gene expression is within the scope of the invention, including a decrease in anywhere from about 1% to 100%. In another aspect of the invention, gene suppression is reversible by treatment with inhibitors, as discussed below.

DNA and/or Histone Methylation

The invention provides compositions and methods for methylation of target sequences. Gene suppression in the methods provided herein can occur by methylation of target sequences and/or by methylation of chromatin-associated proteins, e.g., histone proteins in associated chromatin. Methylation can be complete or partial methylation of the target sequences. DNA methylation can be detected using restriction enzymes and PCR, e.g., as discussed in the Example. Assays known in the art can be used to detect DNA methylation (e.g. bisulfite sequencing) and modification of associated histones (chromatin immunoprecipitation assays).

### Inhibition of Gene Suppression

The invention provides compositions and methods to inhibit gene suppression. In one aspect, treatment of host cells, target sequences, or siRNAs with inhibitors can be used to reduce or block gene silencing. Thus, inhibitors can make gene silencing reversible. In one aspect of the invention, gene silencing inhibitors include trichostatin A (tsa) and 5-azacytidine (5-azaC) or equivalents (see Example).

### Application of Gene Silencing

The invention provides compositions and methods to inhibit gene expression of a target sequence or gene for disease treatment. Genes of interest that can be inhibited using a composition or method of the invention include, but are not limited to, genes associated with autoimmune disease, inflammatory diseases, cancer, influenza, HIV, tumors, neurodegenerative diseases, or any gene that is the focus of research. Thus, the invention also provides siRNAs that target genes associated with autoimmune disease, cancer, influenza, HIV, tumors, neurodegenerative diseases, inflammatory diseases, or any gene that is the focus of research. The methods provided herein may be practiced in vitro, ex vivo or in vivo.

### Disease Treatment

The invention provides compositions and methods to inhibit gene expression related to diseases that arise from the abnormal expression of a particular gene or group of genes. Many conditions have genes associated with them (i.e. a gene is the cause or part of the cause of the condition to be treated). siRNAs of the invention can be used to inhibit the expression of the deleterious gene and therefore alleviate symptoms of a disease or treat disease. siRNA gene silencing of the invention can be used to treat diseases that result from the expression or overexpression of genes. For example, genes contributing to a cancerous state or to viral replication (e.g. HIV viral replication) can be inhibited. Thus, the methods provided herein can be used to silence viral expression.

The methods and compositions provided herein also can be used to target tumor initiation or proliferation of genes. In addition, mutant genes causing dominant genetic diseases such as myotonic dystrophy can be inhibited. Inflammatory diseases such as arthritis can also be treated by inhibiting such genes as cyclooxygenase or cytokines. In addition, siRNAs could be used to generate animals that mimic true genetic “knockout” animals to study gene function. Thus, the methods provided herein can be used to specifically knockout genes. The methods of gene suppression provided herein can be done at the promoter level.

### Gene Screening

The methods and compositions provided herein can be used to screen genes involved in various processes. In one aspect of the invention, high throughput screening can be used to target and suppress gene expression. The methods of gene screening provided here can be used to facilitate basic research.

### Drug Discovery

The methods and compositions of the invention can be used in drug discovery. The siRNA methods and compositions of the invention can be used for target validation; and, in some applications, can provide a quicker and less expensive approach to screen potential drug targets. Information for drug targeting will be gained not only by inhibiting a potential drug target but also by determining whether an inhibited protein, and therefore the pathway, has significant phenotypic effects. For example, inhibition of LDL receptor expression can raise plasma LDL levels and, therefore, suggest that up-regulation of the receptor would be of therapeutic benefit. Expression arrays can be used to determine the responsive effect of inhibition on the expression of genes other than the targeted gene or pathway.

### Treatment of Patients

The gene silencing methods and compositions of the invention can be used to treat patients. In one aspect, a target sequence can be integrated into a patient's genome, and the patient can be treated with target sequence specific siRNAs. Methods (e.g., protocols) and compositions (e.g., formulations) for transfecting cells in vivo are known in the art, as discussed herein.

### Kits

The invention provides kits comprising compositions and instructions for use comprise description of the methods of the invention. The kits can comprise cells, siRNAs, target sequences, transfecting agents, transducing agents, instructions (regarding the methods of the invention), or any combination thereof. As such, kits, cells, and libraries of compounds are provided herein.

The invention will be further described with reference to the following examples; however, it is to be understood that the invention is not limited to such examples.

### EXAMPLES

### Example 1

### Transcriptional Gene Silencing

The following example describes an exemplary method of the invention for RNA-mediated transcriptional regulation—transcriptional down-regulation, or silencing of a gene.

siRNAs were directed against a promoter sequence of a gene target sequence in human cells. The promoter-directed siRNAs inhibited transcription of gene target sequences. While the methods of the invention are not limited by any particular mechanism of action, in this exemplary method silencing of gene target sequences resulted from de novo, in vivo DNA methylation of a target genomic nucleic acid sequence (the target molecule) and/or modification of associated histones, thus providing a means to inhibit mammalian gene function.

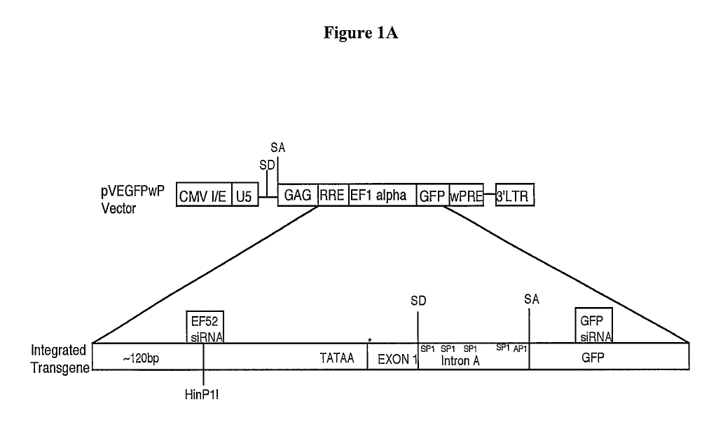

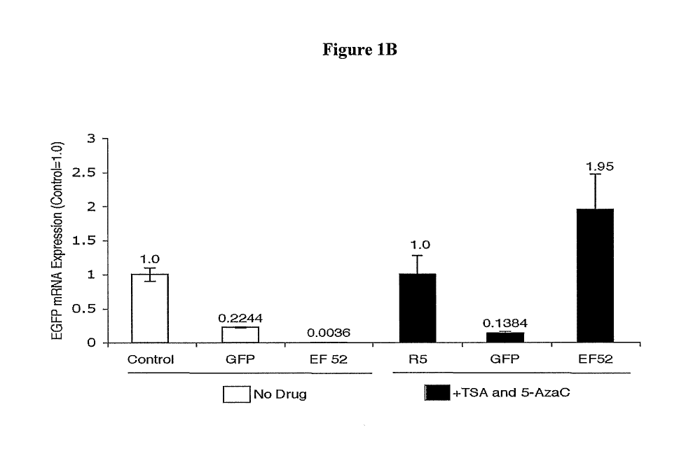

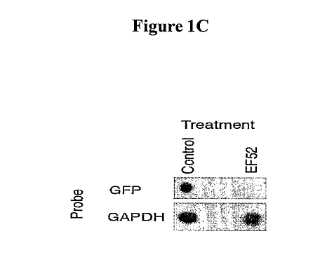

As a target sequence for siRNA-induced transcriptional silencing in human 293FT cells, an EF1A (elongation factor 1 alpha) promoter-GFP (green fluorescent protein) reporter gene integrated into a genome using a lentiviral feline immunodeficiency virus (FIV) vector was used (FIG. 1A) (see, e.g., Morris et al. (2004) “Transduction of cell lines and primary cells by FIV-packaged HIV vectors.” Mol. Ther. 2004 July; 10(1):181-90). siRNA EF52 is homologous to a sequence in the EF1A promoter that has been shown to be involved in transcription using deletion analysis (Wakabayasi-Ito (1994) J. Biol. Chem. 269, 29831-29837). A second siRNA homologous to the GFP coding region in exon 2 was designed to target post-transcriptional mRNA destruction.

FIG. 1A shows lentiviral FIV vector pVE-GFPwP integrating an EF1A promoter-GFP transgene into human cells. siRNA target sites in the promoter (EF52) and coding region (GFP) of the reporter gene are shown. FIG. 1B shows promoter-targeted siRNA inhibits gene expression. 293FT cells were transduced in duplicate with lentivirus and were transfected with control (CCR5), GFP or EF52 siRNAs. GFP mRNA was quantified by real-time RT-PCR. (C) EF52 siRNA silences transcription. Nuclear run-on assay was performed using nuclei from 293FT cells transduced with lentivirus and mock- or EF52 siRNA-transfected.

293FT cells were transduced with an EF1A-GFP vector, and integration was allowed to occur for 24 hours. The 293FT cells were transfected with either EF52, GFP or a control siRNA matching the human CCR5 gene. mRNA and DNA analyses were carried out 48 hours after siRNA transfection for all experiments. The siRNA targeting the GFP mRNA transcript reduced expression relative to the control siRNA as measured by quantitative real-time RT-PCR (FIG. 1B) (see, e.g., Elbashir (2001) Nature 411, 494-8). Inhibition of GFP expression was also seen with the promoter targeting siRNA EF52, indicating that an siRNA directed against a promoter sequence can reduce gene expression in mammalian cells (FIG. 1B, open bars).

### Transcriptional Silencing in Mammalian Cells

Inhibition of GFP expression by promoter-directed siRNAs occurs at the transcriptional level. While the invention cannot be limited by any particular mechanism of action, transcriptional silencing in mammalian cells can be associated with a combination of chromatin modifications that include, but are not limited to, histone deacetylation (and consequently histone methylation) and cytosine DNA methylation (see, e.g., Bird (1999) Cell 99:451-454).

### Inhibition of Transcription—and Reversal of Gene Silencing

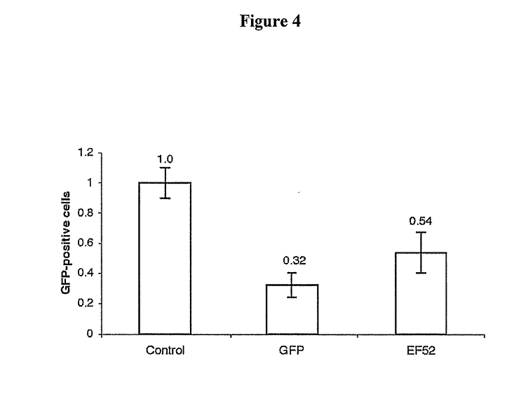

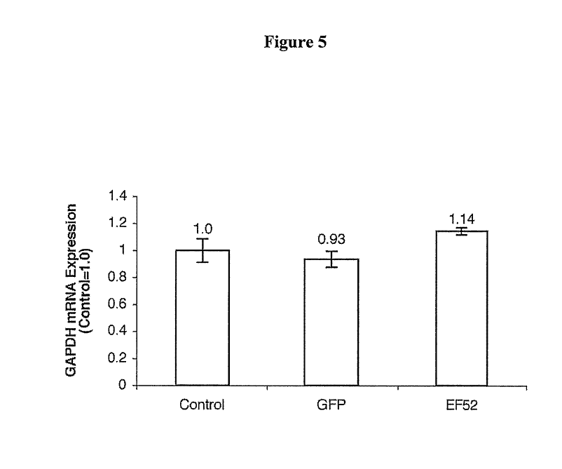

Silencing by EF52 siRNA was completely reversed by treating the cells with trichostatin (TSA) and 5-azacytidine (5-azaC), drugs that inhibit histone deacetylases and DNA methyltransferases respectively (FIG. 1B) (see, e.g., Bird (1999) Cell 99:451-454). These pharmacological inhibitors of transcriptional silencing had no effect on down-regulation caused by RNA interference targeted to the GFP transcript. Gene silencing was confirmed at the transcriptional level by using nuclear run-on analysis, which indicated a 93% reduction in transcriptional initiation from the EF1A-GFP reporter gene in the presence of EF52 siRNA (FIG. 1C). A GAPDH control was unaffected by EF52 siRNA in nuclear run-on and RT-PCR experiments; this and the CCR5 siRNA control showed that promoter-directed transcriptional silencing is specific (FIG. 1B and FIG. 4). The number of GFP positive cells is reduced by EF52 and FDP siRNAs.

### Down-Regulation of Gene Expression

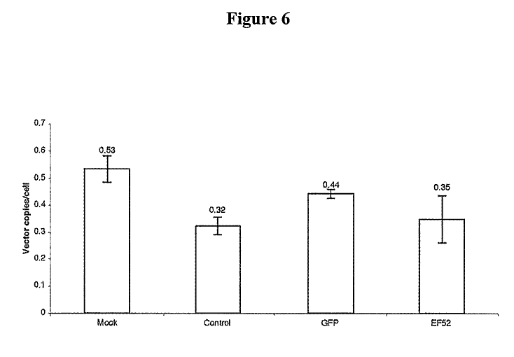

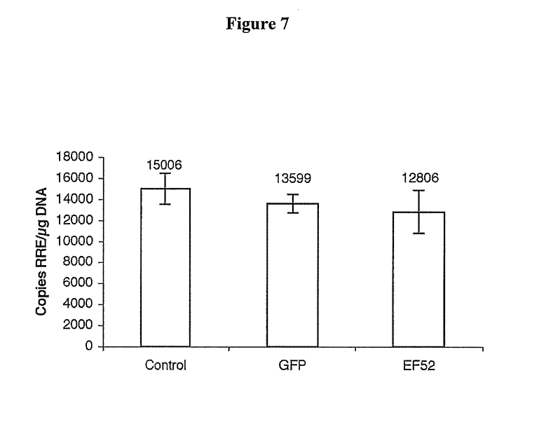

Control experiments confirmed that EF52 siRNA down-regulates EF1A promoter-driven GFP expression by silencing transcription. To ensure that transcription from the EF1A promoter in the integrated transgene initiated in a position corresponding to that of endogenous EF1A, RT-PCR analysis was performed (FIG. 7) (see, e.g., Wakabayasi-Ito (1994) J. Biol. Chem. 269, 29831-29837; Uetsuki (1989) J. Biol. Chem. 264:5791-5798). Specifically, transcripts containing the FIV RRE upstream of the EF1A promoter were not detected, nor were multiple other possible spliced messages initiating from the FIV long-terminal repeat (FIGS. 6 and 7). These results confirmed the transcriptional silencing of the FIV LTR, which has been shown previously to be quiescent (see, e.g., Sauter (2001) Somatic Cell Molecular Genetics 26:99-129), and showed that siRNA EF52 targeted the transgenic EFLA promoter, not a transcribed region. FIG. 6 shows that treatment with siRNA does not alter vector integration. FIG. 7 shows that siRNA transfection does not alter total copies of vector in transduced cells. RRE specific real-time kinetic PCR was performed on genomic DNA from vector transduced siRNA transfected cultures.

While the lentiviral vector used integrated into the chromosome and produced a transcriptionally active transgene within 24 hours (see e.g., Matzke (2001) Science 293:1080-1083; Lund (2004) Science 303, 95-98), PCR analysis of lentiviral DNA was performed to ensure that EF52 siRNA did not interfere with lentiviral transduction. Neither integration frequency nor total lentiviral DNA were affected by siRNA treatment (FIGS. 6 and 7). Collectively, these results demonstrated that EF52 siRNA targeted a promoter region rather than transcribed RNA, did not reduce the number of transgenes, and induced transcriptional gene silencing in human cells.

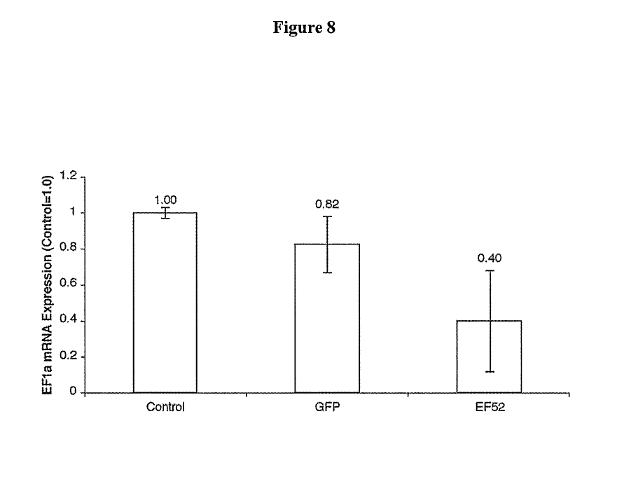

FIG. 8 shows that endogenous EF1a expression is inhibited by EF52 in transduced siRNA transfected 293FT cells. EF1a message was determined by EF1a real-time kinetic RT PCR. The results represent a minimum of 2 experiments with standard deviations shown.

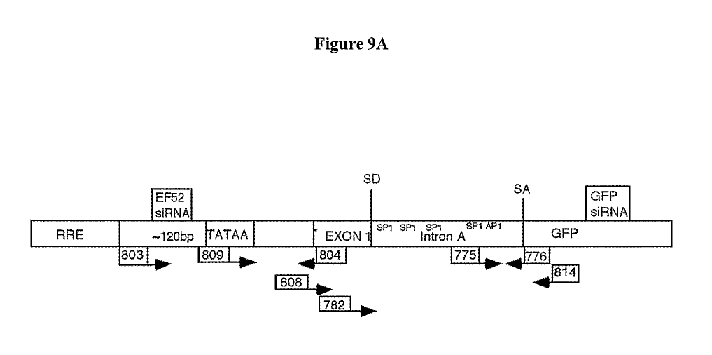

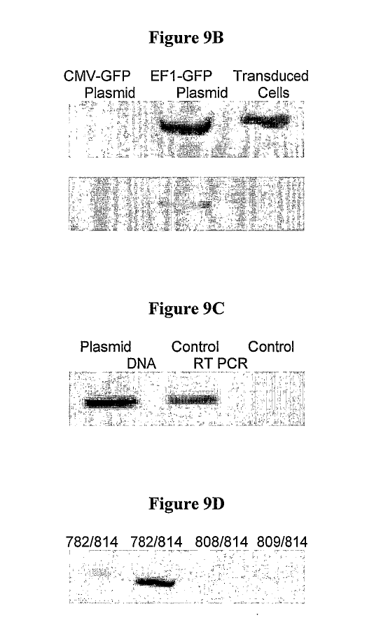

FIG. 9A shows primers used to determine EF1a transcriptional profile relative to various features of the integrated pVE-GFPwP vector and EF1a expressed GFP transgene. FIGS. 9B, 9C and 9D show that integrated vector EF1a promoter expressed GFP transgene is spliced correctly.

### DNA Methylation

Cytosine DNA methylation is a major mediator of gene silencing in mammalian cells, and de novo DNA methylation in plants is guided by small RNAs (see, e.g., Chan (2004) Science 303:1336; Bird (1999) Cell 99:451-454; Matzke (2001) Science 293:1080-1083). The EF52 siRNA target within the EF1A promoter contained a restriction site for the methylation-sensitive enzyme HinP1I. If methylated, this site is protected from digestion, and a PCR product spanning the site can be amplified.

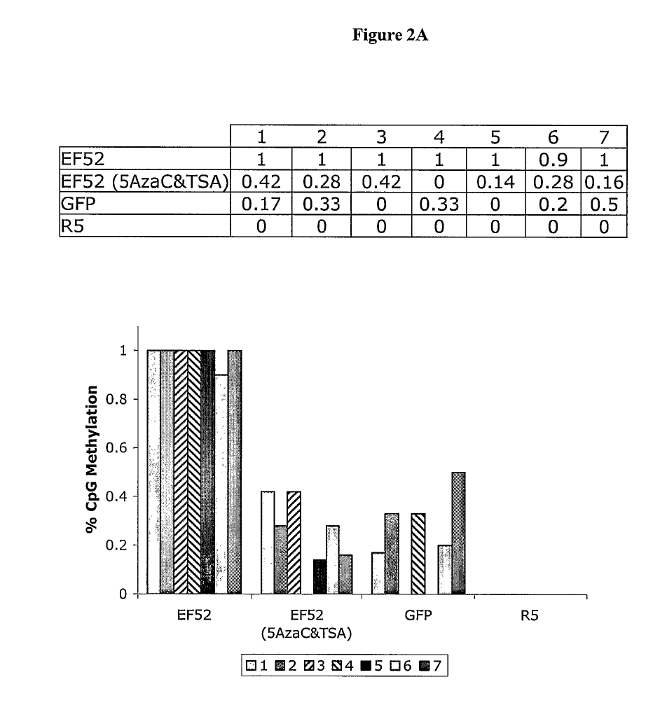

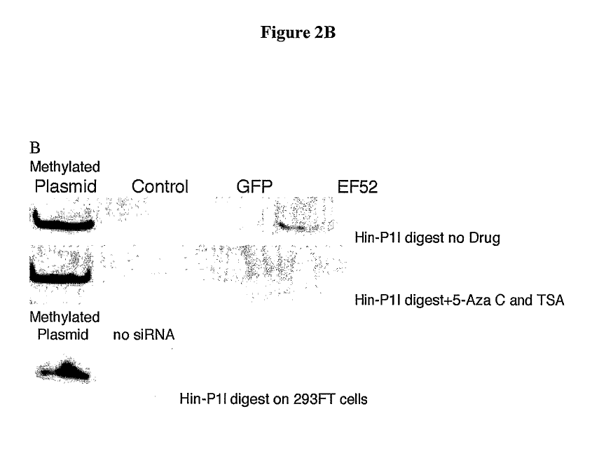

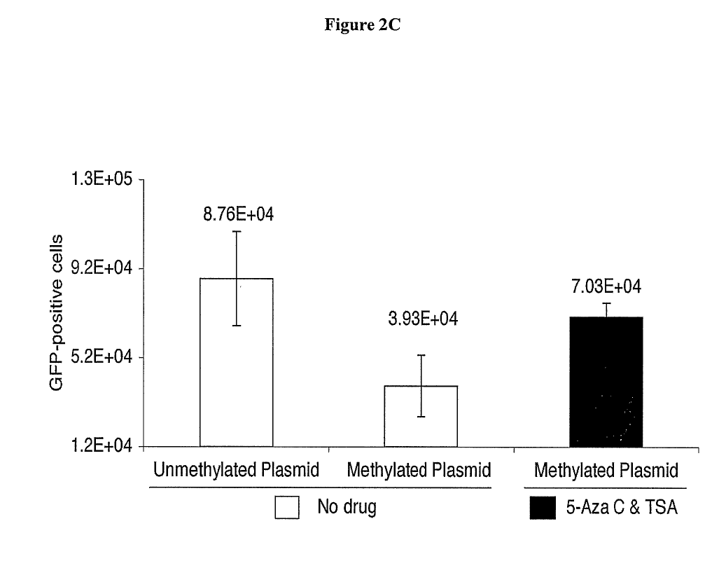

The HinP1I site was unmethylated in genomic DNA from untreated cells or from cells treated with control or GFP siRNAs, but the site was methylated when treated with EF52 promoter-directed siRNA (FIG. 2A; the HinP1I assay measured DNA methylation at the endogenous locus as well as at the EF1A-GFP reporter). Treatment with TSA and 5-azaC reversed DNA methylation and restored expression from a reporter plasmid methylated with bacterial DNA methyltransferase Sss-I (FIG. 2B). Similarly, methylation induced by EF52 siRNA was abolished by treatment with TSA and 5-azaC (FIG. 2A). Detection by bisulfite genomic sequencing revealed that DNA methylation within the EF52 siRNA-targeted sequence was present in EF52 treated cells, but not in cells treated with GFP siRNA (FIG. 2C). Hence, the correlation between DNA methylation and transcriptional inhibition that was seen for other silent loci in mammalian cells was also seen in siRNA-induced transcriptional silencing (see, e.g., Bird (1999) Cell 99:451-454). These results showed that de novo DNA methylation directed by siRNA was conserved between plants and mammals, and the results indicated a molecular mechanism by which siRNA-induced transcriptional silencing occurred in human cells.

FIG. 2A shows de novo DNA methylation guided by siRNA. (A) HinP1I-based DNA methylation assay of the EF1A promoter. DNA was prepared from lentiviral transduced cells transfected with control (CCR5), GFP or EF52 siRNAs (with or without TSA and 5-azaC treatment, top panel), or from untreated 293FT cells (bottom panel). HinP1I cuts within the EF52 siRNA target site, preventing PCR amplification in unmethylated samples. SssI-methylated EF1A promoter-GFP plasmid DNA is a control. FIG. 2B shows TSA and 5-azaC counteract transcriptional inhibition caused by DNA methylation. FACs analysis of GFP expression in cells transfected with SssI-methylated EF1A promoter-GFP plasmid, with or without TSA and 5-azaC treatment. FIG. 2C shows Bisulfite genomic sequencing of the EF1A promoter from an integrated transgene, including the EF52 siRNA target site. Genomic DNA was prepared from lentiviral transduced, siRNA transfected 293FT cells with or without TSA and 5-azaC treatment. The sequenced region contains 9 CG dinucleotides numbered 1 through 9. CG sites 4 and 5 are within the HinP1I site.

Whether or not endogenous EF1A expression was affected by promoter-directed siRNA treatment was examined. However, in mammalian cells that have not been transduced with lentivirus, transfected small RNAs do not have an efficient means of transport into the nucleus, and mammalian cells have specialized export pathways for the hairpin-containing RNA precursors in miRNA biosynthesis (see, e.g., Lund (2004) Science 303:95-8; Yi (2003) Genes Dev. 17:3011-3016; Simeoni (2003) Nucleic Acids Res. 31:2717-2724). This obstacle was circumvented by lentiviral transduction, which permeabilizes the nuclear membrane prior to siRNA transfection (see, e.g., Sherman (2002) Microbes Infect 4:67-73). Therefore, in order to assess the effect of siRNA on the endogenous EF1A promoter, 293FT cells were transfected with EF52 and control (HIV-1 polymerase-specific) siRNAs using MPG, a bipartite amphipathic peptide which incorporates the fusion peptide domain of HIV-1 gp41 and the SV40 nuclear localization sequence (see, e.g., Simeoni (2003) Nucleic Acids Res 31, 2717-24). MPG facilitates nuclear import of nucleic acids, including siRNAs, while fluorescently labeled siRNAs transfected with an MPG variant lacking the nuclear localization sequence are predominantly found in the cytoplasm (see, e.g., Simeoni (2003) Nucleic Acids Res 31, 2717-24).

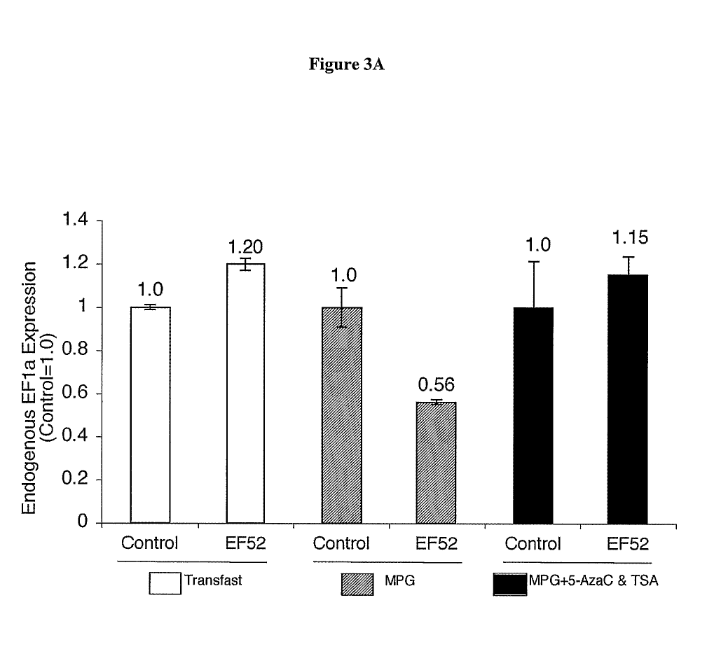

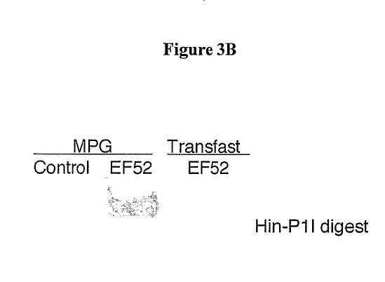

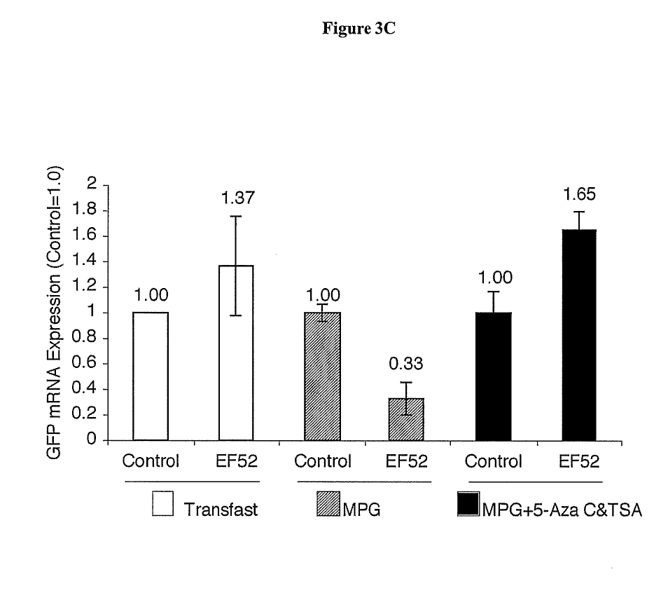

Cells transfected with EF52 and MPG showed a significant reduction of endogenous EF1A expression by real-time RT-PCR (FIG. 3A). Transfection with EF52 using Transfast 48 hours after lentiviral transduction exhibited silencing of endogenous EF1A expression comparable to MPG transfected, untransduced cells. Silencing in cells transfected with EF52+MPG was abolished by treatment with TSA and 5-azaC, indicating that silencing occurred at the transcriptional level (FIG. 3A). Furthermore, HinP1I digestion of the EF1A promoter was blocked in cells treated with EF52+MPG, but not in cells transfected with EF52 using Transfast (FIG. 3B). Thus, siRNAs produced de novo DNA methylation and silencing of the endogenous mammalian promoter.

FIG. 3 (A) shows promoter-targeted siRNA inhibits endogenous EF1A. EF1A expression was determined by real-time RT-PCR in cells transfected with control (HIV-1 polymerase) or EF52 siRNAs using MPG (a nuclear import-mediating peptide) or conventional Transfast reagent. Black columns represent MPG transfected cells treated with TSA and 5-azaC. (B) EF52 siRNA causes de novo DNA methylation of the endogenous EF1A promoter. DNA methylation of the endogenous EF1A promoter was assayed by the HinP1I method in cells that were transfected with control HIV-1 polymerase or EF52 siRNAs using either MPG or Transfast. (C) Nuclear-imported siRNAs inhibit an integrated EF1A promoter-GFP transgene long after lentiviral transduction. Lentiviral transduced cells were sorted for GFP expression, grown for 8 weeks, then transfected as in (B). GFP message was measured by real-time RT-PCR. Results represent 2 experiments with 3 independent samples/experiment.

siRNA Entry Into a Nucleus

The need for nuclear transport of siRNAs in silencing of an integrated EF1A promoter-GFP reporter gene well after transduction was examined (FIG. 3C). For this experiment, 293FT cells were transduced, a GFP-positive population was isolated after 72 hours, and the cells were grown for 8 weeks. As observed for endogenous EF1A, silencing of the integrated EF1A promoter-GFP in this population was dependent on transfection with MPG, and was reversed by TSA and 5-azaC (FIG. 3C). These data indicated that siRNA-induced transcriptional silencing of an integrated reporter was not strictly dependent on the process of lentiviral transduction, but rather on the ability of siRNAs to gain access to the nucleus. Silencing of endogenous EF1A and of the EF1A promoter-GFP reporter gene 8 weeks after transduction was less efficient than siRNA-induced transcriptional silencing observed with newly integrated EF1A promoter-GFP (compare FIGS. 3A and 3C to FIG. 1B). Newly integrated EF1A-GFP transgenes may be more accessible to siRNAs because of their intrinsic chromatin structure. Newly transformed transgenes are more susceptible to de novo DNA methylation and silencing in Arabidopsis (X. Cao, S. E. Jacobsen, Curr Biol 12, 1138-44. (2002)).

The results demonstrated that guidance of de novo DNA methylation by siRNAs is common to plants and animals, indicating that the mechanism for establishing this widespread eukaryotic gene silencing mark has been preserved. By analogy to the DRM methyltransferases of plants, it is possible that de novo DNA methyltransferase enzymes of the Dnmt1 and Dnmt3 family are guided by small RNA in many different biological contexts, including genomic imprinting and establishment of methylation at retroviruses and repeated transgenes (S. W. Chan et al., Science 303, 1336 (2004); X. Cao et al., Curr Biol 13, 2212-7 (2003); Okano (1999) Cell 99:247-257). In one such model, siRNAs might target chromatin-level silencing directly, by binding to either homologous DNA or nascent transcripts and recruiting histone-modifying enzymes and de novo DNA methyltransferases in a manner suggested by the S. pombe RNA-induced transcriptional silencing complex (Grewal (2003) Science 301:798-802; A. Verdel et al., Science 303, 672-6 (2004)).

These results also demonstrate that siRNA transport into the nucleus using the methods of the invention facilitates transcriptional gene silencing in mammalian cells. Transfected siRNAs are generally retained in the cytoplasm of mammalian cells, where they mediate efficient mRNA cleavage, but are incapable of targeting chromatin (Simeoni (2003) Nucleic Acids Res. 31:2717-2724). siRNAs transcribed from hairpin transgenes can be exported from the nucleus, by virtue of their resemblance to pre-miRNAs, which are produced by the nuclear RNaseIII Drosha and cleaved into mature miRNAs by cytoplasmic Dicer (Tijstennan (2002) Annu. Rev. Genet. 36:489-519); and Y. Lee et al., Nature 425, 415-9 (2003)). Transcriptional silencing in mammalian cells may require transfection reagents that transport siRNAs into the nucleus, or siRNA-producing transgenes that do not involve a hairpin intermediate.

While the invention is not limited by any particular mechanism of action, the siRNA-induced transcriptional silencing, e.g., the transcriptional down-regulating methods of the invention, can be more effective than RNA interference for long-term inhibition of gene expression because a single treatment with promoter-targeted siRNA can initiate silencing that is subsequently maintained in the absence of siRNA by DNA methylation and other chromatin modifications. Silencing can be triggered at any time, including well before a particular gene is transcribed. This property is useful when the methods of the invention target genes that are expressed in tissues refractory to siRNA delivery. This property is useful when the methods of the invention are used to permanently silence latent proviruses, such as integrated human immunodeficiency virus (HIV). This property is useful when the methods of the invention are used in the transcriptional silencing of nascently integrated proviruses. Additionally, these data demonstrate that the chromatin-targeted siRNAs of the invention are effective as therapeutic agents for human disease.

### Example 2

### Transcriptional Gene Silencing

The following example describes an exemplary method of the invention for RNA-mediated transcriptional regulation—transcriptional down-regulation, or silencing of a gene.

Using exemplary methods of the invention, a lentiviral vector containing enhanced green fluorescent protein (EGFP) driven by the EF1a promoter was used to demonstrate silencing of expression produced by small interfering RNA (siRNA) directed against EF1a intron A in transfected and transduced human peripheral mononuclear cells and 293T fibroblasts. Short interfering RNA (siRNA (Elbashir et al., 2001; Sharp, 2001) directed against intron A of the EF1 alpha promoter was found to inhibit GFP expression in lentiviral vector transduced siRNA transfected human cells.

### Inhibition of GFP Expression in Transfected Cells

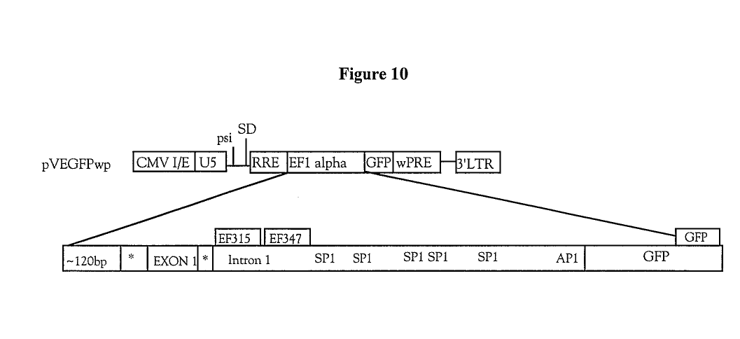

To determine the susceptibility of intron A of the EF1a promoter to siRNA mediated silencing, a panel of siRNAs were designed targeting the EGFP coding region (GFP) and the EF1-alpha intron A (EF315, and 347) (see, e.g., Wakabayasi-Ito, 1994). An siRNA directed against a non-expressed gene CCR5 (CCR5) served as the control. FIG. 10 is an illustration of the EFla promoter and intron A targets. FIV vector pVEGFPwP was packaged by co-transfection with pC34N and VSV-G in 293FT cells (Morris et al., 2004b). EF1-a siRNA target sites (21 bp siRNAs constructed using the AMBION SILENCER™ kit) were EF315 5′-AAG TGG GTG GGA GAG TTC GAG-3′ (SEQ ID NO:1), EF347 5′-AAG GAG CCC CTT CGC CTC GTG-3′ (SEQ ID NO:2), GFP mRNA specific 5′-AAC GAT GCC ACC TAC GGC AAG-3′ (SEQ ID NO:3), and control CCR5 5′-AAT TCT TTG GCC TGA ATA ATT-3′ (SEQ ID NO:4). EF1 alpha promoter is shown with * indicating mRNA start, necessary SP1 and AP1 sites within the EF 1 alpha intron A are also shown (Wakabayasi-Ito, 1994).

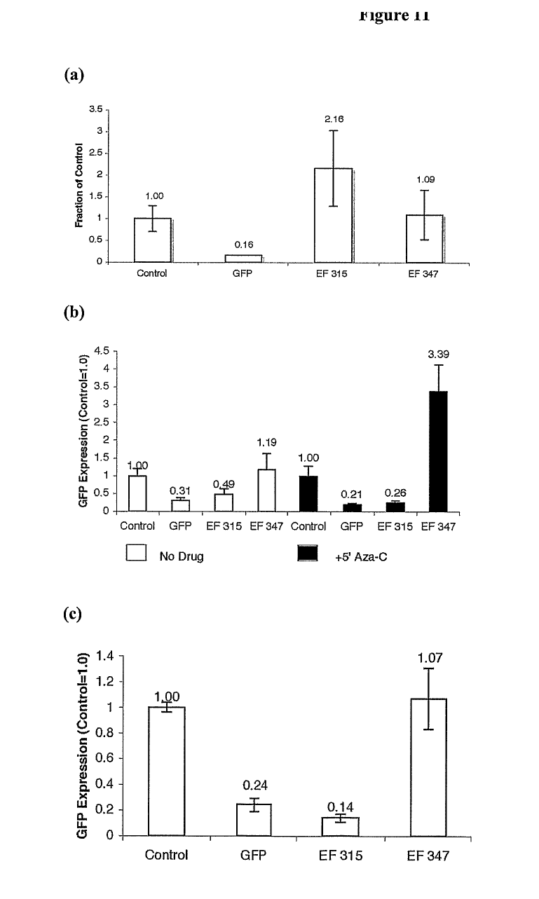

GFP expression in 293FT cells transfected first with vector and 24 hours later by siRNA was reduced significantly by the GFP but not EF315 or EF347 siRNAs (FIG. 11A). FIG. 11A illustrates data showing intron targeted siRNA suppression of GFP using this exemplary method of the invention. Vector pVEGFPwP (1.0 μg) was directly transfected (GENE PORTER 2™, Gene Therapy Systems) into 293FT cells. Twentyfour hours later these cells were transfected with 10 nM of respective siRNAs. Fortyeight hours post-siRNA transfection the RNA was extracted 48, GFP copy number determined by real-time kinetic RT PCR and standardized based on cell numbers.

In cells transfected with vector alone (without FIV Rev) the bulk of transcription is driven from the EGFP cassette rather than the 5′ CMV I/E promoter, though some unspliced transcripts are still produced (Table 1, right), in contrast to the situation in transduced cells, where transcription is driven exclusively from the EF1 alpha cassette (Table 1, left). Table 1 show data comparing RRE to GFP copies/cell in transfected and transduced 293FT cultures.

TABLE 1

| | TD (MOI = | TD (MOI = | TF (1.0 | TF 1.0 |
|---|---|---|---|---|
| Sample | 2.5) RRE | 2.5) GFP | μg) RRE | μg GFP |
| R5 | <0.1 | 21.7 ± 2.2 | 0.5 ± .0.3 | 11.9 ± 3.5 |
| GFP | <0.1 | 4.9 ± 0.2 | 0.3 ± 0.01 | 1.9 ± 0 |
| EF 315 | <0.1 | 3.0 ± 1.6 | 1.7 ± 0.02 | 25.7 ± 10.4 |
| EF 347 | <0.1 | 5.3 ± 1.3 | 0.6 ± 0.3 | 13.0 ± 6.8 |

TD=Transduced cells, TF=Transfected cells, RRE indicates copies of RRE transcript/cell and GFP indicates copies of GFP transcript/cell by real-time kinetic PCR. Real-time kinetic RT PCR for GFP was described previously (FIG. 11A) and for RRE was based on the same protocol with RRE specific primers

(SEQ ID NO:5)

#13 5′-AGATACTTCATCATTCCTCCTCTTTTT-3′,

(SEQ ID NO:6)

#14 5′-TTGATATGGCAATTCCTGCATT-3′,
and

(SEQ ID NO:7)

RRE TET probe 5′-AGGAGAAATGGTAGGCAA-3′

following pre-established protocols (Sheeter, 2003).

### Inhibition of GFP Expression in Transduced Cells

To determine activity of siRNA on intron A from the EF1-alpha promoter in transduced cells, vector was prepared (Morris et al., 2004b), 293FT cells were transduced, and 24 hours later transfected with siRNAs as described previously (Morris et al., 2004a). The EF 315 and GFP siRNA, were found to reduce GFP transcription in transduced (MOI 0.7) 293FT cells (FIG. 11B, open bars) as well as human peripheral blood mononuclear cells (PBMC) (FIG. 11C), demonstrating that siRNAs targeting intronic sequences are active in mammalian cells.

FIG. 11B illustrates data where 293FT cells (1.0×10⁵) were transduced (MOI=0.7) with FIV packaged pVEGFPwP and 24 hrs later transfected with 10 nM of indicated siRNAs. GFP expression was determined by real-time kinetic RT PCR 48 hrs post-siRNA transfection. Shaded boxes represent cultures treated with 4 μM 5′ Aza-C for 48 hours while open boxes represent untreated cultures. FIG. 11C illustrates data where human PBMCs (1.0×10⁴) were transduced with FIV packaged pVEGFPwP (MOI=0.7) and 24 hrs later transfected with 10 nM siRNAs. GFP expression was determined by real-time kinetic PCR 48 hours later. The results (a-c) represent duplicate experiments measured in triplicate.

siRNA Inhibition of GFP Expression is Specific

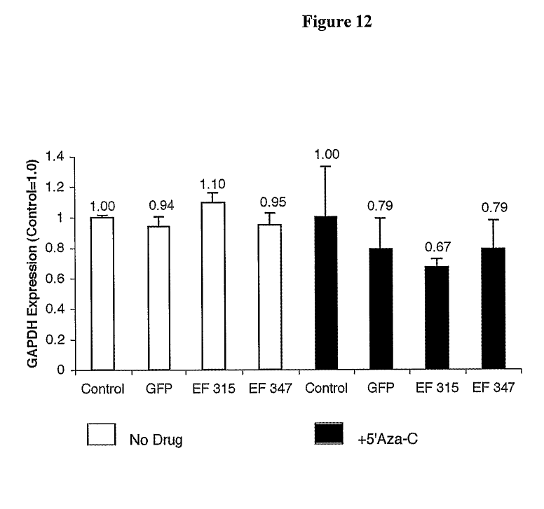

To ensure that inhibition produced by intronic-directed siRNAs was not mediated by non-specific effects on cellular transcription, levels of glutaraldehyde phosphate dehydrogenase mRNA were determined by real-time kinetic PCR in transduced cells (FIG. 12) (Sheeter, 2003). No inhibitory effect on GAPDH mRNA transcription was evident after treatment with any siRNA. The lack of R5 siRNA effect on GFP expression also argues against non-specific effects. Additionally, no increased levels of cell death in siRNA transduced cells was noted (not shown).

FIG. 12 illustrates the specificity of transcriptional inhibition produced by using intron-targeted siRNAs in exemplary methods of the invention. GAPDH expression from 293FT cells (non-drug treated, open columns) and (with 5-Aza C treatment, filled columns) transfected as described previously (FIG. 11B) measured by real-time kinetic RT PCR. The results represent three independent measurements from a single experiment and standard deviations are shown.

The work presented here with vector transduced 293FT and human PBMCs, along with similar findings in yeast (Reinhart & Bartel, 2002; Volpe, 2002), Drosophila (Pal-Bhadra et al., 2002), and C. elegans (Butler, 2001) and in mammals (Kawasaki & Taira, 2004; Morris et al., 2004a), demonstrate that the methods of the invention using RNAi pathways are functional against introns in mammalian cells.

In one aspect, the methods of the invention are specific for targeting the EF1 alpha intron A or other promoters with strongly enhancing intrinsic introns, such as the CMV I/E promoter. The finding that EF1 intron A specific siRNA induced reduction of GFP transgene expression coinciding with lentiviral mediated nuclear delivery is similar to previous observations of TGS in other model systems (Butler, 2001; Pal-Bhadra et al., 2002; Reinhart & Bartel, 2002; Volpe, 2002; Kawasaki & Taira, 2004; Morris et al., 2004a; Kawasaki et al., 2005). While the invention is not limited by any particular mechanism of action, the inability of 5′Aza-C treatment to normalize levels of transcription argue that the observed intron targeted silencing is post-transcriptional gene silencing (PTGS) in nature. However, because the invention is not limited by any particular mechanism of action, in some aspects, the transcriptional down-regulation (e.g., “gene silencing”) effected by the methods of the invention can be either transcriptional gene silencing (TGS), post-transcriptional gene silencing (PTGS) or a combination thereof.

Alternatively, because the invention is not limited by any particular mechanism of action, in some aspects, these siRNAs might be working on or together with small non-coding RNAs, involved in gene regulation, as described for promoters in both human cells and C. elegans (Lagos-Quintana, 2001; Lau, 2001; Lee, 2001). Regardless of the particular mechanism of action, these findings demonstrate that the methods and compositions (e.g., kits) of the invention can be used and are effective in gene promoter or enhancer regulation in vivo. These findings also demonstrate that the methods and compositions (e.g., kits) of the invention can be used and are effective in therapeutic interventions aimed at altering patterns of gene expression.

### Materials and Methods

Vectors, siRNA, Transfection and Transductions—FIV vector pVE-GFPwP was prepared by harvesting supernatant 48 hours after co-transfection (CaPO₄) of pVE-GFPwP (15 μg), pC34N packaging construct (10 μg), and VSV-G expression plasmid (pVSVG, 5 μg) into 4×10⁶ 293FT cells in 10 cm² plates (Morris et al., 2004b). Small interfering RNAs were constructed following established protocols (Ambion SILENCER™). EF 1-alpha siRNA target sites were: EF315 5′-AAG TGG GTG GGA GAG TTC GAG-3′ (SEQ ID NO:1), EF347 5′-AAG GAG CCC CTT CGC CTC GTG-3′ (SEQ ID NO:2), GFP mRNA specific 5′-AAC GAT GCC ACC TAC GGC AAG-3′ (SEQ ID NO:3) (kit control), and negative control CCR5 specific 5′-AAT TCT TTG GCC TGA ATA ATT-3′ (SEQ ID NO:4). Human PBMCs or 293FT cells (1×10⁴ or 1×10⁵, respectively) in duplicate were either transfected with pVE-GFPwP (1 μg) (GENE PORTER 2™, Gene Therapy Systems) or transduced (MOI=0.7) in the presence of 8 μg/ml Polybrene and 24 hrs later these cells were transfected with 10 nM of siRNA as described previously (Morris et al., 2004a). After 48 hrs (72 hours post vector transfection or transduction) the cells were collected and RNA or DNA extracted (Qiagen RNeasy™ and DNeasy™ kits) as described (Morris et al., 2004a).
RT PCR—Cellular RNA was isolated (Qiagen Rneasy™), and 1 μg of RNA was DNase treated (˜5-10 units/μg of RNA, 2 hr) and used as template for the RT reaction (20 μl). RT reaction mix contained 1 μl (2 μM) of primers specific for GFP 814 5′-GGT GGT GCA GAT GAA CTT CAG GGT C-3′ (SEQ ID NO:8), FIV RRE 5′-TTG ATA TGG CAA TTC CTG CAT T-3′ (SEQ ID NO:9), and GAPDH 5′-TGG GAT TTC CAT TGA TGA CAA G-3′ (SEQ ID NO: 10), along with AMV RT (5 U/μl), 2 μl dNTP's (1.25 mM), 2 μl RT buffer (100 mM Tris-HCl (pH 9.0 at 25° C.), 500 mM KCl; 15 mM MgCl₂, 1% Triton X-100 (TX100), 4 μl of MgCl₂ (25 mM), and 0.6 μl of rRNasin (20-40 U/μl). The resultant cDNA (1 hr/42° C. and 10 min/95° C., 50 ng) was then used in subsequent Real-Time Kinetic PCR (described below) or in PCR reactions to determine splice patterns. The 50 μPCR reaction used to determine the pVE-GFPwP transfected and transduced culture splicing patterns consisted of 50 ng of cDNA and was carried out in a mixture of (5 μl 10×PCR buffer, 8 μl dNTP mix, 40 pmol each primer, 1.25 U Taq DNA polymerase, and 37.5 μl sterile Dnase-free water) and amplified (1 cycle-95 C/8 min, 35 cycles 95 C/30 sec, 55 C/30 sec, 72 C/45 sec, followed by terminal extension 1 cycle 72 C/10 min). Primers 673 5′-GGA TGC ATT GAG GAG AAA TGG TAG GC-3′ (SEQ ID NO: 11), 674 5′-GGC AGG CCT ACA ATA CAT ACT TTA TTA G-3′ (SEQ ID NO:12), 803 5′-AAG TGG GGG GAG GGG TCG GCA-3′ (SEQ ID NO:13), 804 5′-GCA CTT ACC TGT GTT CTG GCG GC-3′ (SEQ ID NO:14), 805 5′-GAA AAA AAG AAC GTT CAC GGC GAC TA-3′ (SEQ ID NO:15), 808 5′-GCA GTA GTC GCC GTG AAC GTT C-3′ (SEQ ID NO:16), 809 5′-AAC GGG TTT GCC GCC AGA AC-3′ (SEQ ID NO:17), 775 5′-GGC GCC GTC CAG GCA CCT CGA TTA GTT CT-3′ (SEQ ID NO:18), 776 5′-AAC TTC AGG GTC AGC TTG CCG TAG GTG GCA TCG CC-3′ (SEQ ID NO:19), 775 5′-GGC GCC GTC CAG GCA CCT CGA TTA GTT CT-3′ (SEQ ID NO:20), 776 5′-AAC TTC AGG GTC AGC TTG CCG TAG GTG GCA TCG CC-3′ (SEQ ID NO:21), and 782 5′-TTC GCA ACG GGT TTG CCG CCA GAA C-3′ (SEQ ID NO:22) were used in subsequent RT PCR, and or PCR analyses.
Real-Time Kinetic PCR—Real-time kinetic PCR was run on 50 ng of sample cDNA, or equivalent amount of non-reverse transcribed sample RNA as a control, following previously established protocols (Sheeter, 2003). Primers used for GFP were 625F 5′-AGC AAA GAC CCC AAC GAG AA-3′ (SEQ ID NO:23), 684R 5′-GGC GGC GGT CAC GAA-3′ (SEQ ID NO:24), and 5′-CGC GAT CAC ATG GTC CTG CTG G-3′ (SEQ ID NO:25) 646T (Tet probe), for FIV RRE using primers 13 5′-AGA TAC TTC ATC ATT CCT CCT CTT TTT-3′ (SEQ ID NO:26), 14 5′-TTG ATA TGG CAA TTC CTG CAT T-3′ (SEQ ID NO:27), and RRE TET probe 5′-AGG AGA AAT GGT AGG CAA-3′ (SEQ ID NO:28), and for GAPDH with primers 60R 5′-TGG CAC CGT CAA GGC TGA GAA CG-3′ (SEQ ID NO:29), 59F 5′-TGG GAT TTC CAT TGA TGA CAA G-3′ (SEQ ID NO:30), and FAM probe 5′-CCA CCC ATG GCA AAT TCC-3′ (SEQ ID NO:31), and standardized to cell number from respective samples.

### REFERENCES

Aufsatz W, F. Mette, J. Winden, A. J. M. Matzke, M. Matzke. 2002. RNA-directed DNA methylation in Arabidopsis. Proceedings of the National Academy of Sciences 99:16499-16506.
Bosher J M, P. Dugourcq, S. Sookhareea, M. Labouesse. 1999. RNA Interference Can Target Pre-mRNA: consequences for Gene Expression in a Caenorhabditis elegans Operon. Genetics 153:1245-1256.
Butler S L, M. S. T. Hansen, F. D. Bushman. 2001. A quantitative assay for HIV DNA integration in vivo. Nature Medicine 7:631-634.
Elbashir S M, Harborth J, Lendeckel W, Yalcin A, Weber K, Tuschl T. 2001. Duplexes of 21-nucleotide RNAs mediate RNA interference in cultured mammalian cells. Nature 411:494-498.
Fire A, S. Xu, M. K. Montgomery, S. A. Kostas, S. E. Driver, C. C. Mello. 1998. Potent and specific genetic interference by double stranded RNA in Caenorhabditis elegans. Nature 391:806-811.
Jones L, F. Ratcliff, D. C. Baulcombe. 2001. RNA-directed transcriptional gene silencing in plants can be inherited independently of the RNA trigger and requires Met1 for maintenance. Current Biology 11:747-757.
Kawasaki H, Taira K. 2004. Induction of DNA methylation and gene silencing by short interfering RNAs in human cells. Nature.
Kawasaki H, Taira K, Morris K V. 2005. siRNA Induced Transcriptional Gene Silencing in Mammalian Cells. Cell Cycle 4.
Lagos-Quintana M, R. Rauhut, W. Lendeckel, T. Tuschl. 2001. Identification of Novel genes coding for small expressed RNAs. Science 294:853-857.
Lau N C, L. P. Lim, E. G. Weinstein, D. P. Bartel. 2001. An abundant class of tine RNAs with probable regulatory roles in Caenorhabditis elegans. Science 294.
Lee R C, V. Ambros. 2001. An extensive class of small RNAs in Caenorhabditis elegans. science 94:862-864.
Liu J, Carmell M A, Rivas F V, Marsden C G, Thomson J M, Song J J, Hammond S M, Joshua-Tor L, Hannon G J. 2004. Argonaute2 Is the Catalytic Engine of Mammalian RNAi. Science.
Mette M F, A. J. M. Matzke, and M. A. Matzke. 2001. Resistance of RNA-mediated TGS to HC-Pro, a viral suppressor of PTGS, suggests alternative pathways for dsRNA processing. Current Biology 11:11119-1123.

Mette M F, W. Aufsatz, J. Van der Winden, A. J. M. Matzke, and M. A. Matzke. 2000. Transcriptional silencing and promoter methylation triggered by double-stranded RNA. The EMBO Journal 19:5194-5201.
Montgomery M K, S. Xu, A. Fire. 1998. RNA as a target of double-stranded RNA-mediated genetic interference in Caenorhabditis elegans. Proceedings of the National Academy of Sciences 95:15502-15507.
Morris K V, Chan S W, Jacobsen S E, Looney D J. 2004a. Small interfering RNA-induced transcriptional gene silencing in human cells. Science 305:1289-1292.
Morris K V, Gilbert J, Wong-Staal F, Gasmi M, Looney D J. 2004b. Transduction of cell lines and primary cells by FIV-packaged HIV vectors. Mol Ther 10:181-190.
Ngo H, C. Tschudi, K. Gull, E. Ullu. 1998. Double-stranded RNA induces mRNA degradation in Trypanosoma brucei. Proceedings of the National Academy of Sciences 95:14687-14692.
Nishikura K. 2001. A short primer on RNAi: RNA-directed RNA polymerase acts as a key catalyst. Cell 107:415-418.
Pal-Bhadra M, Bhadra U, Birchler J A. 2002. RNAi related mechanisms affect both transcriptional and posttranscriptional transgene silencing in Drosophila. Mol Cell 9:315-327.
Pal-Bhadra M, Leibovitch B A, Gandhi S G, Rao M, Bhadra U, Birchler J A, Elgin S C. 2004. Heterochromatic silencing and HP1 localization in Drosophila are dependent on the RNAi machinery. Science 303:669-672.
Reinhart B J, Bartel D P. 2002. Small RNAs correspond to centromere heterochromatic repeats. Science 297:1831.
Robb G B, Brown K M, Khurana J, Rana T M. 2005. Specific and potent RNAi in the nucleus of human cells. Nat Struct Mol Biol 12:133-137.
Sharp P A. 2001. RNA interference. Genes and Development 15:485-490.
Sheeter D, P. Du, S. Rought, D. Richman, J. Corbeil. 2003. Surface CD4 expression Modulated by a Cellular Factor Induced by HIV type 1 Infection. AIDS Research and Human Retroviruses 19:117-123.
Sijen T, I. Vign, A. Rebocho, R. Blokland, D. Roelofs, J. Mol, and J. Kooter. 2001. Transcriptional and posttranscriptional gene silencing are mechansitically related. Current Biology 11:436-440.
Volpe T A, C. Kidner, I. M. Hall, G. Teng, S. I. S. Grewal, R. A. Martienssen. 2002. Regulation of Heterchromatic Silencing and Histone H3 Lysine-9 Methylation by RNAi. Science 297:1833-1837.
Wakabayasi-Ito N, and S. Nagata. 1994. Characterization of the Regulatory Elements in the Promoter of the Human Elongation Factor-1 alpha Gene. The Journal of Biological Chemistry 269:29831-29837.
Wassenegger M. 2000. RNA-directed DNA methylation. Plant Molecular Biology 43:203-220.
Wassenegger M, M. W. Graham, M. D. Wang. 1994. RNA-directed de novo methylation of genomic sequences in plants. Cell 76:567-576.
Zeng Y, B. R. Cullen. 2002. RNA interference in human cells is restricted to the cytoplasm. RNA July 8:855-860.

A number of embodiments of the invention have been described. Nevertheless, it will be understood that various modifications may be made without departing from the spirit and scope of the invention. Accordingly, other embodiments are within the scope of the following claims.

1. A method of silencing or down-regulating gene transcription comprising:
(1) (i) (a) identifying a target nucleic acid sequence in a gene of a cell;
(b) providing a target sequence specific short interfering RNA (siRNA);
(c) transfecting the target specific siRNA into a nucleus of the cell, thereby silencing or down-regulating gene transcription; and
(d) monitoring transcriptional suppression in the cell; or
(ii) (a) providing a target sequence specific short interfering RNA (siRNA), wherein the target sequence is complementary to a gene sequence in an animal cell; and
(b) importing the target specific siRNA into a nucleus of the animal cell, thereby silencing or down-regulating gene transcription;
(2) the method of (1), wherein importing the target specific siRNA into the nucleus comprises permeabilization of the nuclear envelope:
(3) the method of (2), wherein permeabilization of the nuclear envelope is effected by a lentiviral co-transduction;
(4) the method of (1), wherein the transfecting of the target specific siRNA into a nucleus comprises use of a nuclear active transport mechanism;
(5) the method of (4), wherein the nuclear active transport mechanism comprises a nuclear-transport mediating composition;
(6) the method of (4) or (5), wherein the nuclear active transport mechanism or nuclear-transport mediating composition comprises an importin β (karyopherin β), an SV40 T antigen nuclear localization signal, a human LEDGF/p75 protein, NLSV404, a tetramer of NLSV404, MPG, a nucleoporin protein or an active fragment thereof or a combination thereof;
(7) the method of (1), wherein the target nucleic acid sequence comprises a transcriptional regulatory sequence and the siRNA comprises a sequence complementary to the transcriptional regulatory sequence;
(8) the method of (7), wherein the transcriptional regulatory sequence comprises a promoter sequence and the siRNA comprises a sequence complementary to the promoter sequence;
(9) the method of (7), wherein the transcriptional regulatory sequence comprises an enhancer sequence and the siRNA comprises a sequence complementary to the enhancer sequence;
(10) the method of (1), wherein the siRNA modifies gene transcription of the target gene;
(11) the method of (10), wherein the siRNA partially silences, or down-regulates, transcription of the target gene, thereby effecting at least partial silencing of the gene;
(12) the method of (10), wherein the siRNA completely silences, or down-regulates, transcription of the target gene, thereby effecting complete silencing of the gene;
(13) the method of (10), wherein partial or complete transcriptional suppression occurs prior to transcription and generation of mRNA;
(14) the method of (1), wherein the at least partial silencing of the gene is reversible;
(15) the method of (1), further comprising methylation of the target sequence;
(16) the method of (1), wherein the target sequence comprises a marker or reporter gene;
(17) the method of (1), wherein the cell is a mammalian cell;
(18) the method of (17), wherein the mammalian cell is a human cell;
(19) the method of (1), further comprising amplification of the target sequence;
(20) the method of (19), wherein the amplification comprises polymerase chain reaction amplification (PCR);
(21) the method of (1), wherein the siRNA is 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or 10 or fewer nucleotides in length;
(22) the method of (21), wherein the siRNA is 19 to 25 nucleotides in length;
(23) the method of (22), wherein the siRNA is 21 to 23 nucleotides in length;
(24) the method of (1), wherein at least a portion of the siRNA comprises a double-stranded RNA;
(25) the method of (24), wherein the at least partially double-stranded siRNA comprises two separate oligonucleotides;
(26) the method of (25), wherein the at least partially double-stranded siRNA comprises a folded oligonucleotide;
(27) the method of (24), wherein the double-stranded RNA comprises a single stranded nucleotide overhang;
(28) the method of (27), wherein the single stranded nucleotide overhang comprises a 3′ single stranded nucleotide overhang;
(29) the method of (27), wherein the single stranded nucleotide overhang comprises a two or three nucleotide 3′ overhang;
(30) the method of (1), wherein the siRNA comprises a triple helix-forming oligonucleotide that specifically binds to a double-stranded DNA sequence;
(31) the method of (1), wherein the siRNA comprises a synthetic, non-natural or modified RNA;
(32) the method of (31), wherein the synthetic, non-natural or modified RNA comprises 2′-O-methyl-containing ribonucleotide, a phosphorothioate-containing ribonucleotide, 2′-0-(2-methoxyethyl)-modified oligonucleotide, or a combination thereof;
(33) the method of (31), wherein the synthetic, non-natural or modified RNA comprises a phosphodiester, a phosphorothioate or a 2′-O-methyl phosphodiester oligonucleotide, or a combination thereof;
(34) the method of any of (1) to (31), wherein the target sequence is complementary to at least a portion of a sequence of a infectious agent;
(35) the method of (34), wherein the infectious agent is a DNA virus or a retrovirus;
(36) the method of (35), wherein the DNA virus is an Epstein-Barr virus (EBV), cytomegalovirus (CMV), Rhesus monkey rhadinovirus (RRV), Kaposi's sarcoma-associated herpesvirus (KSHV), parvovirus B19 (B19), varicella-zoster virus (VZV), and/or any or the herpesvirus, e.g., human herpesvirus (HHV)-1, (HHV)-2, (HHV)-5, (HHV)-6, (HHV)-7, or human herpesvirus 8 (HHV-8);
(37) the method of (35), wherein the retrovirus is a lentivirus; or
(38) the method of (37), wherein the lentivirus is a human immunodeficiency virus (HIV), or the lentivirus is HIV-1 or HIV-2.

2. (canceled)

3. A method for in vivo DNA methylation of a gene, an associated chromatin or a combination thereof comprising:
(1) (a) providing a target sequence specific short interfering RNA (siRNA), wherein the target sequence is complementary to a gene sequence in an animal cell, and the animal cell comprising genomic nucleic acid comprising the target sequence; and
(b) importing the target specific siRNA into the nucleus of the animal cell, thereby effecting in vivo DNA methylation of the gene target sequence or associated chromatin;
(2) the method of (1), wherein importing the target specific siRNA into the nucleus comprises permeabilization of the nuclear envelope;
(3) the method of (2), wherein permeabilization of the nuclear envelope is effected by a lentiviral co-transduction;
(4) the method of (1), wherein the transfecting of the target specific siRNA into a nucleus comprises use of a nuclear active transport mechanism;
(5) the method of (4), wherein the nuclear active transport mechanism comprises a nuclear-transport mediating composition;
(6) the method of (4) or (5), wherein the nuclear active transport mechanism or nuclear-transport mediating composition comprises an importin β (karyopherin β), an SV40 T antigen nuclear localization signal, a human LEDGF/p75 protein, NLSV404, a tetramer of NLSV404, MPG, a nucleoporin protein or an active fragment thereof or a combination thereof;
(7) the method of (1), wherein the target nucleic acid sequence comprises a transcriptional regulatory sequence and the siRNA comprises a sequence complementary to the transcriptional regulatory sequence;
(8) the method of (7), wherein the transcriptional regulatory sequence comprises a promoter sequence and the siRNA comprises a sequence complementary to the promoter sequence;
(9) the method of (7), wherein the transcriptional regulatory sequence comprises an enhancer sequence and the siRNA comprises a sequence complementary to the enhancer sequence;
(10) the method of (1), wherein the siRNA modifies gene transcription of the target gene;
(11) the method of (10), wherein the siRNA partially silences, or down-regulates, transcription of the target gene, thereby effecting at least partial silencing of the gene;
(12) the method of (10), wherein the siRNA completely silences, or down-regulates, transcription of the target gene, thereby effecting complete silencing of the gene;
(13) the method of (10), wherein partial or complete transcriptional suppression occurs prior to transcription and generation of mRNA;
(14) the method of (1), wherein the at least partial silencing of the gene is reversible;
(15) the method of (1), further comprising methylation of the target sequence;
(16) the method of (1), wherein the target sequence comprises a marker or reporter gene;
(17) the method of (1), wherein the cell is a mammalian cell;
(18) the method of (17), wherein the mammalian cell is a human cell;
(19) the method of (1), further comprising amplification of the target sequence;
(20) the method of (19), wherein the amplification comprises polymerase chain reaction amplification (PCR);
(21) the method of (1), wherein the siRNA is 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or 10 or fewer nucleotides in length;
(22) the method of (21), wherein the siRNA is 19 to 25 nucleotides in length;
(23) the method of (22), wherein the siRNA is 21 to 23 nucleotides in length;
(24) the method of (1), wherein at least a portion of the siRNA comprises a double-stranded RNA;
(25) the method of (24), wherein the at least partially double-stranded siRNA comprises two separate oligonucleotides;
(26) the method of (25), wherein the at least partially double-stranded siRNA comprises a folded oligonucleotide;
(27) the method of (24), wherein the double-stranded RNA comprises a single stranded nucleotide overhang;
(28) the method of (27), wherein the single stranded nucleotide overhang comprises a 3′ single stranded nucleotide overhang;
(29) the method of (27), wherein the single stranded nucleotide overhang comprises a two or three nucleotide 3′ overhang;
(30) the method of (1), wherein the siRNA comprises a triple helix-forming oligonucleotide that specifically binds to a double-stranded DNA sequence;
(31) the method of (1), wherein the siRNA comprises a synthetic, non-natural or modified RNA;
(32) the method of (31), wherein the synthetic, non-natural or modified RNA comprises 2′-O-methyl-containing ribonucleotide, a phosphorothioate-containing ribonucleotide, 2′-0-(2-methoxyethyl)-modified oligonucleotide, or a combination thereof;
(33) the method of (31), wherein the synthetic, non-natural or modified RNA comprises a phosphodiester, a phosphorothioate or a 2′-O-methyl phosphodiester oligonucleotide, or a combination thereof;
(34) the method of any of (1) to (31), wherein the target sequence is complementary to at least a portion of a sequence of a infectious agent;
(35) the method of (34), wherein the infectious agent is a DNA virus or a retrovirus;
(36) the method of (35), wherein the DNA virus is an Epstein-Barr virus (EBV), cytomegalovirus (CMV), Rhesus monkey rhadinovirus (RRV), Kaposi's sarcoma-associated herpesvirus (KSHV), parvovirus B19 (B19), varicella-zoster virus (VZV), and/or any or the herpesvirus, e.g., human herpesvirus (HHV)-1, (HHV)-2, (HHV)-5, (HHV)-6, (HHV)-7, or human herpesvirus 8 (HHV-8);
(37) the method of (35), wherein the retrovirus is a lentivirus; or
(38) the method of (37), wherein the lentivirus is a human immunodeficiency virus (HIV), or the lentivirus is HIV-1 or HIV-2.

4-35. (canceled)

36. A method of methylating a genomic target sequences in an animal cell, comprising:
(i) (a) identifying a target sequence in a genome of an animal cell;
(b) providing a target sequence specific siRNA;
(c) transfecting the target specific siRNA into a nucleus of the animal cell, wherein the transfection results in methylation of the target sequence; and
(d) analyzing the methylated target sequences, or
(ii) the method of (i), further comprising amplification of the target sequence.

37. A method of methylating target sequences comprising:
(i) (a) providing a target sequence specific siRNA, wherein the target sequence is complementary to a gene sequence in the animal cell; and
(b) importing the target specific siRNA into a nucleus of the animal cell or
(ii) the method of (i), further comprising amplification of the target sequence.

38. (canceled)

39. A method of inhibiting gene silencing in a cell comprising:
(i) (a) identifying a target sequence in a genome of the cell;
(b) providing a target sequence specific siRNA;
(c) transfecting the target specific siRNA into the nucleus of animal cell;
(d) treating the animal cell with gene silencing inhibitors; and
(e) monitoring inhibition of gene suppression in the animal cell;
(ii) the method of (i), further comprising amplification of the target sequence; or
(iii) the method of (i) or (ii), wherein the gene silencing inhibitors comprise trichostatin A and 5-azacytidine.

40. (canceled)

41. A method of ameliorating or treating a disease or a condition in a subject by transcriptional silencing comprising:
(i) (a) administering to the subject a composition comprising a siRNA that targets a gene of interest, wherein transcriptional silencing of the gene of interest treats or ameliorates the disease or condition; and
(b) importing the gene targeting siRNA into the nucleus of a cell of the subject, wherein the cell comprises the gene of interest, thereby silencing expression of the gene of interest and ameliorating the disease or condition;
(ii) method of (i), wherein the method partially or completely silences transcription of the gene of interest; or
(ii) method of (i), further comprising integrating the gene of interest into a subject genome.

42-43. (canceled)

44. A method of drug discovery comprising:
(i) (a) identifying a target gene sequence in a genome of an animal cell;
(b) providing a gene target sequence specific siRNA,
(c) importing the gene target specific siRNA into the nucleus of the animal cell, wherein the siRNA suppresses target gene expression; and
(d) interacting a test agent with the animal cell or siRNA to determine which test agent inhibits or promotes gene silencing; or
(ii) the method of (i), wherein the animal cell is a mammalian or human cell.

45. A method for transcriptionally silencing a latent provirus comprising
(i) (a) providing a target sequence-specific short interfering RNA (siRNA), wherein the target sequence is complementary to at least one gene of a latent provirus or a retrovirus gene sequence integrated into the genome of an animal cell; and
(b) importing the target specific siRNA into a nucleus of the animal cell;
(ii) the method of (i), wherein the integrated provirus or retrovirus comprises an integrated retrovirus or lentivirus;
(ii) the method of (i), wherein the integrated lentivirus comprises a human immunodeficiency virus (HIV);
(iii) the method of (ii), wherein the HIV is HIV-1 or HIV-2;
(iv) the method of (i), wherein the target sequence is complementary to an LTR of the latent provirus or retrovirus; or
(v) the method of (iv), wherein the LTR comprises an HIV-1 LTR.

46-48. (canceled)

49. A method for transcriptionally silencing a DNA virus comprising
(i) (a) providing a target sequence-specific short interfering RNA (siRNA), wherein the target sequence is complementary to at least a partial sequence of a DNA virus; and
(b) importing the target specific siRNA into a nucleus of the animal cell; or
(ii) the method of (i), wherein the target sequence is complementary to a sequence of Epstein-Barr virus (EBV), cytomegalovirus (CMV), Rhesus monkey rhadinovirus (RRV), Kaposi's sarcoma-associated herpesvirus (KSHV), parvovirus B19 (B19), varicella-zoster virus (VZV), and/or any or the herpesvirus, e.g., human herpesvirus (HHV)-1, (HHV)-2, (HHV)-5, (HHV)-6, (HHV)-7, or human herpesvirus 8 (HHV-8).

50. (canceled)

51. A kit for transcriptionally silencing a latent provirus or retrovirus, the kit comprising'
(a) siRNA or nucleic acid encoding siRNA, and instructions comprising instructions for practicing the method of claim 1;
(b) the kit of (a), further comprising at least one construct expressing a short sense RNA, a single stranded RNA, a nuclear RNA, a small nuclear RNA, an RNA naturally retained in the nucleus, a short hairpin RNA transcript designed to saturate or overcome nuclear export, and/or an antisense RNA strand; or
(c) the kit of (b), wherein the at least one construct expresses short sense and antisense RNA strands and expresses them separately, in tandem, embedded within a transcript designed to be retained in the nucleus or in short hairpin RNA transcripts designed to saturate or overcome nuclear export

52. A kit for transcriptionally silencing a gene, the kit comprising
(a) siRNA or nucleic acid encoding siRNA, and instructions comprising practicing the method of claim 1;
(b) the kit of (a), further comprising at least one construct expressing a short sense RNA, a single stranded RNA, a nuclear RNA, a small nuclear RNA, an RNA naturally retained in the nucleus, a short hairpin RNA transcript designed to saturate or overcome nuclear export, and/or an antisense RNA strand; or
(c) the kit of (b), wherein the at least one construct expresses short sense and antisense RNA strands and expresses them separately, in tandem, embedded within a transcript designed to be retained in the nucleus or in short hairpin RNA transcripts designed to saturate or overcome nuclear export

53. A kit for the targeted methylation of a gene or associated chromatin, the kit comprising
(a) siRNA, nucleic acid encoding siRNA, and instructions comprising instructions for practicing the method of claim 1;
(b) the kit of (a), further comprising at least one construct expressing a short sense RNA, a single stranded RNA, a nuclear RNA, a small nuclear RNA, an RNA naturally retained in the nucleus, a short hairpin RNA transcript designed to saturate or overcome nuclear export, and/or an antisense RNA strand; or
(c) the kit of (b), wherein the at least one construct expresses short sense and antisense RNA strands and expresses them separately, in tandem, embedded within a transcript designed to be retained in the nucleus or in short hairpin RNA transcripts designed to saturate or overcome nuclear export.

54-60. (canceled)
